(19) United States
(12) Patent Application Publication
(10) Pub. No.: US 2014/0147873 A1
(43) Pub. Date: May 29, 2014

(54) SURFACE DISPLAY OF CELLULOLYTIC ENZYMES AND ENZYME COMPLEXES ON GRAM-POSITIVE MICROORGANISMS

(71) Applicants: Robert T. Clubb, Culver City, CA (US); Timothy Anderson, St. Louis, MO (US)

(72) Inventors: Robert T. Clubb, Culver City, CA (US); Timothy Anderson, St. Louis, MO (US)

(73) Assignee: THE REGENTS OF THE UNIVERSITY OF CALIFORNIA, Oakland, CA (US)

(21) Appl. No.: 14/002,670

(22) PCT Filed: Feb. 29, 2012
(86) PCT No.: PCT/US12/27150

§ 371 Date: Aug. 30, 2013

Related U.S. Application Data
(60) Provisional application No. 61/449,007, filed on Mar. 3, 2011.

(51) Int. Cl. C12P 19/14; C12P 19/02; C12Q 1/34

(52) U.S. Cl. CPC C12P 19/14; C12Q 1/34; C12P 19/02
USPC 435/18; 435/252.1; 435/252.7; 435/252.5; 435/99; 435/289.1; 536/23.2; 435/320.1

(57) ABSTRACT

In various embodiments a system is provided that displays heterologous proteins on the surface of a Gram-positive microorganism. In certain embodiments the system displays proteins using a sortase transpeptidase to covalently anchor proteins to the cell wall of the microbe. Novel bacterial strains are provided to exploit this system to display cellulase enzymes and multi-enzyme complexes on the surface of Gram-positive microorganisms (e.g., Bacillus subtilis) through their non-covalent interaction with a scaffoldin protein that is covalently anchored to the cell wall by the sortase transpeptidase. The surface displayed protein complexes contain enzymes capable of degrading cellulose into its component sugars at accelerated rates as compared to solutions of purified enzymes.

### CROSS-REFERENCE TO RELATED APPLICATIONS

This application claims benefit of and priority to U.S. Ser. No. 61/449,007, filed on Mar. 3, 2011, which is incorporated herein by reference in its entirety for all purposes.

### STATEMENT OF GOVERNMENTAL SUPPORT

This invention was made with Government support under Grant No DE-FC02-02ER63421 awarded by the Department of Energy. The Government has certain rights in this invention.

### BACKGROUND

Dwindling supplies of petroleum have intensified the search for improved methods to produce ethanol from biomass (Kerr (2008) Science 322:1178-9). A limiting step in this process is the degradation of lignocellulose into its component sugars (Himmel et al. (2007) Science 315: 804-7; Margeot et al. (2009) Curr. Opin. Biotechnol. 20(3): 372-80; Zhang et al. (2006) Biotechnol. Adv. 24: 452-481). Lignocellulose is the main component of biomass and consists of cellulose and hemicellulose carbohydrate fibers that are coated with lignin (Harris and DeBolt (2010) Plant Biotechnol. J. 8: 244-262). Although a variety of cellulolysis processes have been demonstrated, commonly used methods first pre-treat lignocellulosic materials with chemicals and/or heat (Hendriks and Zeeman (2009) Bioresour. Technol. 100:10-18; Kim et al. (2009) Meth. Mol. Biol. 581: 79-91; Kim et al. (2009) Meth. Mol. Biol., 581: 93-102; Zhao et al. (2009) Appl. Microbiol. Biotechnol. 82: 815-827). The cellulose is then hydrolyzed into simple sugars by exposing it to a variety of purified cellulases (Miller and Blum (2010) Environ. Technol. 31: 1005-1015; Wilson (2009) Curr. Opin. Biotechnol. 20: 295-299; Yeoman et al. (2010) Adv. Appl. Microbiol. 70: 1-55). An alternative approach that may improve the efficiency of enzymatic degradation is to employ bacterial cellulosomes, multi-cellulase containing complexes that exhibit extremely potent cellulolytic activity. Thus, ongoing research has concentrated on understanding the molecular basis of their cellulolytic activity and sought to engineer cellulosomes for industrial purposes (Doi (2008) Ann. N.Y. Acad. Sci. 1125: 267-279; Fontes and Gilbert (2010) Annu. Rev. Biochem. 79: 655-681; Maki et al. (2009) Int. J. Biol. Sci. 5: 500-516).

Anaerobic meso- and thermophilic-bacteria produce cellulosomes that have a common overall architecture in which a central scaffoldin protein coordinates the binding of different cellulolytic enzymes (Fontes and Gilbert (2010) Annu. Rev. Biochem. 79: 655-681). The cellulosome from Clostridium thermocellum is archetypal (Bayer et al. (1985) J. Bacteriol. 163: 552-559). Its scaffoldin, CipA, has binding sites for nine enzymes (Salamitou et al. (1994) J. Bacteriol. 176: 2822-2827; Tokatlidis et al. (1991) FEBS Lett. 291: 185-188). Binding is mediated by cohesin domains within CipA that interact with sub-nanomolar affinity with dockerin domains that are fused to the cellulolytic enzymes (Miras et al. (2002) Biochemistry 41: 2115-2119; Schaeffer et al. (2002) Biochemistry 41:2106-2114. CipA also contains a cellulose binding domain (CBD), also referred to as a carbohydrate-binding module (CBM), that tethers the cellulosome complex to its substrate as well as a type II dockerin domain located at its C-terminus that anchors the complex to the cell wall by interacting with the peptidylglycan associated SdbA protein (Fontes and Gilbert (2010) Annu. Rev. Biochem. 79: 655-681). A variety of cellulases with distinct activities are incorporated into the cellulosome: endoglucanases, exoglucanases, β-glucosidases, xylanases, and pectinases among others. Enzyme co-localization and the presence of the substrate targeting CBD within the cellulosome enables cultures of C. thermocellum displaying these complexes to decompose cellulose at significantly faster rates than purified enzyme solutions (Lu et al. (2006) Proc. Natl. Acad. Sci., USA, 103: 16165-16169). The specific enzyme composition within the cellulosome is presumably varied to degrade different types of plant matter as the C. thermocellum genome encodes more than sixty dockerin containing enzymes (Ciruela et al. (1998) FEBS Lett. 422: 221-224; Zverlov et al. (2005) Proteomics 5: 3646-3653). Several other species of anaerobic bacteria also degrade cellulose using cellulosomes that contain the same basic architecture constructed from cohesin-dockerin domain interactions.

To exploit their potent cellulolytic activity several research groups have created minicellulosome complexes in which a cohesin containing miniscaffoldin coordinates the binding of dockerin-cellulase fusion proteins (Bayer et al. (1994) Trends Biotechnol. 12: 379-386; Caspi et al. (2008) J. Biotechnol. 135: 351-357; Fierobe et al. (2002) J. Biol. Chem., 277: 49621-49630; Fierobe et al. (2005) J. Biol. Chem., 280: 16325-16334). Because the cohesin-dockerin interaction is species specific, cohesins from different bacterial species are typically used to construct the miniscaffoldin (Haimovitz et al. (2008) Proteomics 8: 968-979; Pages et al. (1997) Proteins 29: 517-527). Ordered and unique multi-protein complexes can then be formed by adding chimeric fusion proteins in which the cellulase enzyme is fused to the appropriate dockerin domain. The enzymatic properties of a number of purified designer minicellulosomes have been characterized in vitro and the cellulolytic activity of different combinations of endoglucanases, exoglucanases, and β-glucosidases have been tested (Caspi et al. (2008) J. Biotechnol. 135: 351-357; Fierobe et al. (2002) J. Biol. Chem. 277: 49621-49630; Fierobe et al. (2005) J. Biol. Chem. 280: 16325-16334; Mingardon et al. (2007) Appl. Environ. Microbiol. 73: 7138-7149). Even the geometry of the miniscaffoldin protein, altering a linear scaffoldin for one that is circular or rectangular in architecture, has been manipulated to determine the effect of enzyme positioning on cellulolytic activity (Mingardon et al. (2007) Appl. Environ. Microbiol. 73: 7138-49). Combined, this work has produced complexes with more potent and synergistic activity against crystalline cellulose as compared to the isolated enzymes, but the complexes were less active than naturally occurring cellulolytic cells (Fierobe et al. (2002) J. Biol. Chem. 277: 49621-30; Fierobe et al. (2005) J. Biol. Chem. 280: 16325-16334; Matsuoka et al. (2007) J. Bacteriol. 189: 7190-7194; Mingardon et al. (2007) Appl. Environ. Microbiol. 73: 7138-7149).

Recently Saccharomyces cerevisiae strains have been created that display designer minicellulosomes. These strains are a step towards the construction of a consolidated bioprocessing microorganism that could produce high levels of ethanol directly from biomass (Lilly et al. (2009) FEMS Yeast Res. 9: 1236-1249; Tsai et al. (2009) Appl. Environ. Microbiol. 75: 6087-6093; Wen and Zhao (2010) Appl. Environ. Microbiol. 76: 1251-1260). To display the minicellulosome on the surface each group covalently linked it to the β-1,6-glucan within the cell wall using a glycosyl phosphatidylinositol (GPI) signal motif. Volshenk and colleagues displayed a miniscaffoldin protein containing two cohesin domains by fusing it to the GPI signal motif from the Cwp2 protein (Lilly et al. (2009) FEMS Yeast Res. 9: 1236-1249). The minicellulosome was then successfully assembled by incubating the yeast with distinct dockerin-cellulase fusion proteins. A slightly different approach was used by the Chen and Zhao groups (Tsai et al. (2009) Appl. Environ. Microbiol. 75: 6087-6093; Wen and Zhao (2010) Appl. Environ. Microbiol. 76: 1251-1260). In this work the Aga1 protein was first covalently anchored to the cell wall via its GPI anchor. Miniscaffoldin proteins containing the Aga2 protein that interacts with Aga1 were then tethered to the cell surface via non-covalent interactions. After incubating the yeast with purified dockerin-cellulase fusion proteins the Chen group successfully assembled uni-, bi-, and tri-cellulase containing minicellulosomes on the cell surface producing a yeast strain that could produce ethanol from cellulose.

### SUMMARY OF THE INVENTION

In various embodiments a system is provided that displays heterologous proteins on the surface of a Gram-positive microorganism. In certain embodiments the system displays proteins using a sortase transpeptidase to covalently anchor proteins to the cell wall of the microbe. Novel bacterial strains are provided to exploit this system to display cellulase enzymes and multi-enzyme complexes on the surface of Gram-positive microorganisms (e.g., Bacillus subtilis) through their non-covalent interaction with a scaffoldin protein that is covalently anchored to the cell wall by the sortase transpeptidase. The surface displayed protein complexes contain enzymes capable of degrading cellulose into its component sugars at accelerated rates as compared to solutions of purified enzymes.

It is believed the B. subtilis display system described herein is particularly well suited for industrial applications because B. subtilis has a robust genetic system and is already used in industry.

In certain embodiments a recombinant Gram-positive bacterium that displays on its surface one or more cellulolytic enzymes is provided. In certain embodiments the bacterium comprises (1) a protein comprising one or more cellulolytic enzyme domains covalently linked to the surface of the microorganism, and a nucleic acid construct that encodes the protein and one or more cellulolytic enzymes attached to a secretory signal sequence at the N-terminus of the protein and a cell wall sorting signal at the carboxyl terminus of the protein; and/or (2) a protein comprising one or more cohesin domains covalently linked to the surface of the bacterium, where the bacterium, comprises a nucleic acid construct that encodes the protein comprising the one or more cohesin domains attached to a secretory signal sequence at the N-terminus of the protein and a cell wall sorting signal at the carboxyl terminus of the protein. In certain embodiments the cell wall sorting signal comprises an LPXTG (SEQ ID NO:14) motif where X is any amino acid. In certain embodiments the cell wall sorting signal comprises a cell wall sorting signal from a Gram-positive microorganism that encodes an endogenous sortase enzyme. In certain embodiments the cell wall sorting signal comprises a cell wall sorting signal from an organism selected from the group consisting of S. aureus, S. sobrinus, E. faecalis, S. pyogenes, L. monocytogenes, A. viscosus, S. agalactiae, S. aureus, S. mutans, and S. pyogenes. In certain embodiments the cell wall sorting signal comprises a domain of the Staphylococcus aureus Fibronectin Binding Protein B. In certain embodiments the cell wall sorting signal comprises an amino acid sequence selected from the group consisting of

(SEQ ID NO: 1)
LPETGGEESTNNGMLFGGLFSILGLALLRRNKKNHKA,
(SEQ ID NO: 2)
LPETGEENPFIGTTVFGGLSLALGAALLAGRRREL,
(SEQ ID NO: 3)
LPETGGEESTNKGMLFGGLFSILGLALLRRNKKNHKA,
(SEQ ID NO: 4)
LPATGDSSNAYLPLLGLVSLTAGFSLLGLRRKQD,
(SEQ ID NO: 5)
LPKTGEKQNVLLTVVGSLAAMLGLAGLGFKRRKETK,
(SEQ ID NO: 6)
LPSTGSIGTYLFKAIGSAAMIGAIGIYIVKRRKA,
(SEQ ID NO: 7)
LPTTGDSDNALYLLLGLLAVGTAMALTKKARASK,
(SEQ ID NO: 8)
LPLTGANGVIFLTIAGALLVAGGAVVAYANKRRHVAKH,
(SEQ ID NO: 9)
LPYTGVASNLVLEIMGLLGLIGTSFIAMKRRKS,
(SEQ ID NO: 10)
LPKTGMKIITSWITWVFIGILGLYLILRKRFNS,
(SEQ ID NO: 11)
LPSTGEQAGLLLTTVGLVIVAVAGVYFYRTRR,
and
(SEQ ID NO: 12)
LPSTGETANPFFTAAALTVMATAGVAAVVKRKEEN.

In various embodiments the bacterium is a bacterium that encodes an endogenous sortase transpeptidase. In certain embodiments the endogenous sortase transpeptidase is upregulated. In certain embodiments the bacterium further comprises a nucleic acid construct encoding a sortase transpeptidase. In certain embodiments the sortase transpeptidase is a sortase A enzyme or a homologue thereof. In certain embodiments the sortase A enzyme is a Bacillus anthracis sortase A enzyme or a homologue thereof. In certain embodiments the sortase transpeptidase and/or a homologue thereof is under control of an inducible promoter.

In certain embodiments the secretory signal sequence comprises a secretory signal sequence from the B. subtilis PhrC protein. In certain embodiments the secretory signal sequence comprises the amino acid sequence MKLKSKLFVICLAA AAIFTAAGVS ANAE ALDFHVT (SEQ ID NO:13).

In certain embodiments the nucleic acid encoding the sortase transpeptidase is under control of a constitutive promoter or an inducible promoter. In certain embodiments the nucleic acid construct that encodes the protein and one or more cellulolytic enzymes attached to a secretory signal sequence at the N-terminus of the protein and a cell wall sorting signal at the carboxyl terminus of the protein is under control (operably linked) to a constitutive promoter or to an inducible promoter. In certain embodiments the nucleic acid construct that encodes the protein comprising the one or more cohesin domains attached to a secretory signal sequence at the N-terminus of the protein and a cell wall sorting signal at the carboxyl terminus of the protein is under control (operably linked) to a constitutive promoter or to an inducible promoter. In certain embodiments any of the nucleic acid constructs described herein integrates into the genome of the host microorganism. In certain embodiments any of the nucleic acid constructs described herein does not integrate into the genome of the host microorganism.

In various embodiments one or more endogenous proteases of the bacterium are down-regulated or knocked out. In certain embodiments the down-regulated or knocked out proteins is a cell wall protease (e.g., WprA protease or a homologue thereof).

In certain embodiments the bacterium comprises a protein comprising one or more cellulolytic enzymes covalently linked to the surface of the microorganism, and a nucleic acid construct that encodes the protein and one or more cellulolytic enzymes attached to a secretory signal sequence at the N-terminus of the protein and a cell wall sorting signal at the carboxyl terminus of the protein. In certain embodiments the bacterium comprises a protein comprising one or more cohesin domains covalently linked to the surface of the microorganism, where the bacterium, comprises a nucleic acid construct that encodes the protein comprising the one or more cohesin domains attached to a secretory signal sequence at the N-terminus of the protein and a cell wall sorting signal at the carboxyl terminus of the protein. In certain embodiments one or more of the cohesin domains are attached to one or more proteins comprising a dockerin attached to one or more cellulolytic enzymes where the dockerin is bound to the one or more cohesin domains. In certain embodiments the protein comprising a dockerin attached to a cellulolytic enzyme further comprises a cellulose or carbohydrate binding domain (CBD). In certain embodiments the cohesin domain(s) comprise a cohesin domain from a Clostridium sp. In certain embodiments the cohesin domain(s) comprise a cohesin domain from Clostridium thermocellum, and/or Clostridium cellulolyticum, and/or Ruminococcus flavefaciens, and/or C. cellulovorans, and/or C. acetobutylicum, and/or C. josui, and/or C. papyrosolvens, and/or A. cellulolyticus, and/or R. albus. In certain embodiments the dockerin attached to a cellulolytic enzyme is encoded by a construct in the bacterium. In certain embodiments the dockerin attached to a cellulolytic enzyme provided from a source extrinsic to the bacterium. In certain embodiments the cellulolytic enzyme(s) on dormant bacteria are stable for at least 1 day, more preferably for at least 2 days, and most preferably at least 3 days.

In various embodiments the displayed cellulolytic enzyme(s) comprise one or more enzymes selected from the group consisting of an endocellulase, an exocellulase, a beta-glucosidase (cellobiase), an oxidative cellulase, a xylanase, a hemicellulase, a lichenase, a chitenase, and a cellulose phosphorylase. In certain embodiments a plurality of cellulolytic enzymes are present/displayed forming a minicellulosome. In certain embodiments the minicellulosome comprises at least 2, more preferably at least 3, still more preferably at least 4, or at least 5, or at least 6, or at least 7, or at least 8, or at least 9, or at least 10 different enzymes. In certain embodiments the minicellulosome comprises at least one endoglucanase. In certain embodiments the minicellulsome comprises at least one exoglucanase. In certain embodiments the minicellulosome comprises at least two endoglucanases and at least one exoglucanase. In certain embodiments the minicellulosome comprises Clostridium cellulolyticum endoglucanase Cel5A, C. cellulolyticum endoglucanase Cel48F, and C. cellulolyticum exoglucanase Cel9E. In certain embodiments the Gram-positive bacterium comprises a Gram-positive bacterium that encodes a sortase. In certain embodiments the Gram-positive bacterium comprises a Gram-positive bacillus. In certain embodiments the Gram-positive bacterium comprises a genus selected from the group consisting of Corynebacterium, Clostridium, Listeria, and Bacillus. In certain embodiments the bacterium is a Clostridium acetobutylicum. In certain embodiments the Gram-positive bacterium is B. subtilis. In certain embodiments the Gram-positive bacterium comprises a thermophilic Geobacillus spp. In certain embodiments the Gram-positive bacterium comprises a Gram-positive coccus. In certain embodiments the bacterium is selected from the group consisting of S. aureus, S. epidermis, and S. saprophyticus. Certain embodiments are not limited to the use of Gram-positive bacteria. In certain embodiments in any Gram-positive microbe that contains a conventional cell wall can be utilized. In certain embodiments any microbe that contains a sortase and a cell wall to which the sortase couples a peptide can be utilized.

In various embodiments methods of degrading cellulosic biomass into fermentable sugars are provided. The methods typically involve contacting the cellulosic biomass with a bacterium displaying one or more cellulolytic enzymes (or minicellulosomes) as described herein under conditions in which the bacteria partially or fully degrade cellulose in the cellulosic biomass to form one or more fermentable sugars. In certain embodiments the contacting comprises contacting dormant bacteria to the cellulosic biomass. In certain embodiments the contacting comprises culturing the bacteria with the cellulosic biomass. In certain embodiments the cellulosic biomass comprise one or more materials selected from the group consisting of an agricultural plant waste (e.g., corn stover, cereal straw, sugarcane bagasse), a plant waste from an industrial process (e.g., sawdust, paper pulp), a non-food energy crop (e.g., switchgrass). In certain embodiments the cellulosic biomass comprises one or more materials selected from the group consisting of grasses, rice hulls, bagasse, jute, hemp, flax, bamboo, sisal, abaca, straw, corn cobs, corn stover, alfalfa, hay, coconut hair, seaweed, and algae.

In various embodiments a consolidated bioreactor for the conversion of a lignocellulosic biomass into biofuel (e.g., lipid-based molecules, alcohol-based molecules, and the like). The bioreactor typically comprises a culture system that cultures bacteria displaying one or more cellulolytic enzymes (or minicellulosomes) as described herein under conditions in which the bacteria partially or fully degrade cellulose in the lignocellulosic biomass to form one or more fermentable sugars; and a culture system that ferments the sugars to form a biofuel.

In various embodiments isolated nucleic acids (or nucleic acid constructs) are provided. The nucleic acids typically comprise a nucleic acid that encodes a protein comprising one or more cellulolytic enzymes attached to a secretory signal sequence at the N-terminus of the protein and a cell wall sorting signal at the carboxyl terminus of the protein; and/or a protein comprising one or more cohesin domains attached to a secretory signal sequence at the N-terminus of the protein and a cell wall sorting signal at the carboxyl terminus of the protein. In certain embodiments the encoded protein further comprises a carbohydrate binding domain. In certain embodiments the nucleic acid encodes a protein comprising one or more cellulolytic enzymes attached to a secretory signal sequence at the N-terminus of the protein and a cell wall sorting signal at the carboxyl terminus of the protein. In certain embodiments the nucleic acid encodes a protein comprising one or more cohesin domains attached to a secretory signal sequence at the N-terminus of the protein and a cell wall sorting signal at the carboxyl terminus of the protein. In certain embodiments the nucleic acid encodes a cell wall sorting signal comprising an LPXTG (SEQ ID NO:14) motif where X is any amino acid. In certain embodiments the nucleic acid encodes a cell wall sorting signal comprising a cell wall sorting signal from a Gram-positive microorganism that encodes an endogenous sortase enzyme. In certain embodiments the nucleic acid encodes aa cell wall sorting signal comprising a cell wall sorting signal from an organism selected from the group consisting of S. aureus, S. sobrinus, E. faecalis, S. pyogenes, L. monocytogenes, A. viscosus, S. agalactiae, S. aureus, S. mutans, and S. pyogenes. In certain embodiments the nucleic acid encodes a cell wall sorting signal comprising a domain of the Staphylococcus aureus Fibronectin Binding Protein B. In certain embodiments the nucleic acid encodes a cell wall sorting signal comprising an amino acid sequence selected from the group consisting of LPETGGEESTNNGMLFGGLFSILGLALLRRNKKNHKA (SEQ ID NO:1), LPETGEENPFI GTTVFGGLSLALGAALLAGRRREL (SEQ ID NO:2), LPETGGEES TNKGMLF GGLF S I LGLALLRRNKKNHKA (SEQ ID NO:3), LP AT GDS SNAYLPLLGLVS LT AGFS LLGLRRKQD (SEQ ID NO:4), LP KTGEKQNVLLTVVGS LAAMLGLAGLGFKRRKETK (SEQ ID NO:5), LP S TGS I GT YLF KAI GS AAMI GAI GI YI VKRRKA (SEQ ID NO:6), LPTTGDSDNALYLLLGLLAVGTAMALT KKARAS K (SEQ ID NO:7), LPLTGANGVI FLTI AGALLVAGGAVVAYANKRRHVAKH (SEQ ID NO:8), LPYTGVAS NLVLEI MGLLGLI GTS F I AMKRRKS (SEQ ID NO:9), LPKTGMKI I TS WI TWVF I GI LGLYLI LRKRFNS (SEQ ID NO: 10), LPSTGEQAGLLLTTVGLVI VAVAGVYF YRTRR (SEQ ID NO: 11), and LP S TGETANPFFTAAALTVMATAGVAAVVKRKEEN (SEQ ID NO: 12). In certain embodiments the nucleic acid encodes a secretory signal sequence comprising a secretory signal sequence from the B. subtilis PhrC protein. In certain embodiments the nucleic acid encodes a secretory signal sequence comprising the amino acid sequence MKLKSKLFVICLA AAAI FTAAGVS ANAE ALDFHVT (SEQ ID NO:13). In various embodiments any of these nucleic acid sequences is provided operably linked to a promoter and optionally in an expression cassette and/or a vector. In certain embodiments the promoter is a constitutive promoter or an inducible promoter. In certain embodiments the promoter is endogenous to a Gram-positive bacterium. In certain embodiments the promoter is endogenous to the bacterium into which the vector is to be placed.

In various embodiments methods of identifying cellulolytic enzyme combinations that enhance degradation of a particular substrate (e.g., biomass) are provided. In certain embodiments the methods involve providing a plurality of recombinant bacteria as described herein, wherein the bacteria each display at least two cellulolytic enzymes and different bacteria display different enzymes; contacting the substrate with the bacteria; and selecting bacteria that show enhanced degradation of the substrate and/or improved growth on the substrate. Also provided is a method of identifying cellulolytic enzyme variants that enhance degradation of a particular substrate. This method typically involves providing a plurality of recombinant bacteria as described herein, wherein said bacteria each display at least one cellulolytic enzyme variant and different bacteria display different cellulolytic enzyme variants; contacting said substrate with said bacteria; and selecting bacteria that show enhanced degradation of the substrate and/or improved growth on the substrate. In certain embodiments the cellulolytic enzyme(s) and/or the cellulolytic enzyme variants comprise a mutant cellulolytic enzyme. In certain embodiments the mutant cellulolytic enzyme comprises a mutant cellulase. In certain embodiments the selecting comprises selecting bacteria that show improved growth on said substrate.

### DEFINITIONS

The term “nucleic acid” refers to a nucleotide polymer, and unless otherwise limited, includes known analogs of natural nucleotides that can function in a similar manner (e.g., hybridize) to naturally occurring nucleotides. The term nucleic acid includes any form of DNA or RNA, including, for example, genomic DNA; complementary DNA (cDNA), which is a DNA representation of mRNA, usually obtained by reverse transcription of messenger RNA (mRNA) or by amplification; DNA molecules produced synthetically or by amplification; and RNA. The term nucleic acid encompasses double- or triple-stranded nucleic acid, as well as single-stranded molecules. In double- or triple-stranded nucleic acids, the nucleic acid strands need not be coextensive (i.e., a double-stranded nucleic acid need not be double-stranded along the entire length of both strands). The term nucleic acid also encompasses any chemical modification thereof, such as by methylation and/or by capping. Nucleic acid modifications can include addition of chemical groups that incorporate additional charge, polarizability, hydrogen bonding, electrostatic interaction, and functionality to the individual nucleic acid bases or to the nucleic acid as a whole. Such modifications may include base modifications such as 2′-position sugar modifications, 5-position pyrimidine modifications, 8-position purine modifications, modifications at cytosine exocyclic amines, substitutions of 5-bromo-uracil, backbone modifications, unusual base pairing combinations such as the isobases isocytidine and isoguanidine, and the like.

The terms “isolated”, when referring to an isolated nucleic acid or nucleic acid construct refers to a nucleic acid that either does not exist normally in nature, and/or that is constructed using for example, recombinant DNA techniques, and/or that is removed from nucleic acid sequences that would normally flank it in vivo, and/or that is removed from a cellular milieu. Isolated nucleic acids also include nucleic acids derived from the foregoing isolated nucleic acids, e.g., by propagation of a construct/vector/organism/virus/or microorganism containing such nucleic acid sequences.

“Operably linked” means that a gene (or other sequence to be expressed) and transcriptional regulatory sequence(s) are connected in such a way as to permit expression of the gene under control of the regulatory sequence(s).

“Exogenous” means a nucleic acid sequence that has been inserted into a host cell or a nucleic acid sequence or amino acid sequence derived from a nucleic acid sequence that has been inserted into a host cell. This includes introduced (inserted) nucleic acids that remain into the cytoplasm and introduced nucleic acids that integrate into the host cell genome (e.g., plasmids inserted into the host genome) as well as nucleic acid sequences and/or amino acids sequences derived from such. In certain embodiments an exogenous sequence can result from the cloning of a native gene from a host cell and the reinsertion of that sequence back into the host cell. In most instances, exogenous sequences are sequences that are derived synthetically, or from cells that are distinct from the host cell.

The terms “host cells” and “recombinant host cells” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “cellulolytic enzyme” refers to an enzyme that can participate in the degradation of cellulose or a cellulosic biomass.

The term “cellulosic biomass” refers to plant, algal, or other biomass that contains cellulose.

Lignocellulosic biomass refers to plant biomass that typically contains cellulose, hemicellulose, and lignin. The carbohydrate polymers (cellulose and hemicelluloses) are often tightly bound to the lignin. Lignocellulosic biomass can be grouped into four main categories: (1) agricultural residues (including corn stover and sugarcane bagasse), (2) dedicated energy crops, (3) wood residues (including sawmill and paper mill discards), and (4) municipal paper waste. Illustrative lignocellulosic biomass sources include, but are not limited to grasses, rice hulls, bagasse, jute, hemp, flax, bamboo, sisal, abaca, straw, corn cobs, corn stover, alfalfa, hay, coconut hair, seaweed, algae,

A cellulase is an enzyme that breaks down cellulose, especially in the wall structures, and a “cellulosome” is an array, cluster, or sequence of enzymes or cellulases that degrades cellulose. In various embodiments cellulosomes comprise catalytic subunits such as glycoside hydrolases, polysaccharide lyases and carboxyl esterases bound together by scaffoldins consisting of cohesins connected to other functional units such as the enzymes and carbohydrate binding modules via dockerins.

A “protein encoding one or more cellulolytic enzymes” or a “protein comprising one or more cellulolytic” refers to a protein at least a portion of which displays cellulolytic activity. In certain embodiments the protein comprises a single cellulolytic enzyme and substantially the entire protein (absent processing and/or signaling sequences) comprises a single enzyme (e.g., a cellulase). In certain embodiments the protein comprises multiple (e.g., 2, 3, 4, 5, 6, or more) cellulolytic enzymes and in such instances each enzyme comprises a different “domain” in said protein. Similarly a protein comprising or encoding multiple cohesins refers to a protein comprising one or more domains each of which has the amino acid sequence of a cohesin, and in certain embodiments, is capable of binding to a corresponding dockerin.

When a Markush Group is described in the specification and/or claims it is intended that in various additional or alternative any subset of that Markush group is contemplated. Thus, for example, a Markush group consisting of elements A, B, and C also comprises a disclosure of a Markush Group consisting of A, and B, a Markush Group consisting of B, and C, and a Markush Group consisting of A and C as well as elements A, B, and C individually.

Where a range of values is provided, it is understood that each intervening value between the upper and lower limit of that range and any other stated or intervening value in that stated range, is contemplated. The upper and lower limits of these smaller ranges may independently be included in the smaller ranges, and are also contemplated, subject to any specifically excluded limit in the stated range. Where the stated range includes one or both of the limits, ranges excluding either or both of those included limits are also contemplated.

### BRIEF DESCRIPTION OF THE DRAWINGS

FIG. 1 illustrates the sortase transpeptidase reaction. The protein intended to be anchored to the surface of the cell contain specific sequences that direct the protein to be secreted from the cell (signal peptide) on the N-terminus, and a cell wall sorting signal (CWSS) on its C-terminus that is recognized by sortase. As the surface protein is being secreted from the cell, sortase will recognize the LPXTG (SEQ ID NO:14) motif within the CWSS and cleave the peptide bond between the T and G, forming a covalent intermediate between the surface protein and sortase. Sortase will then form a peptide bond between the surface protein and the pentaglycine crossbridge (Gly₅ (SEQ ID NO:15)) in Staphylococcus aureus or diaminopimelic acid (DAP) in Bacillus anthracis and Bacillus subtilis within Lipid II. This new peptide bond formation will cause the surface protein to be covalently attached to the cell wall peptidoglycan and cause the protein to be displayed on the surface of the cell.

FIGS. 2A and 2B illustrates surface display of individual enzymes (FIG. 2A) or cellulolytic complexes (FIG. 2B) using Sortase A. As illustrated in this figure sortase transpeptidase SrtA from B. anthracis can be used to display a single cellulase (CelA). Within CelA there is a CWS located within the C-terminus that is recognized by SrtA. When SrtA expression is induced with xylose and CelA expression is induced with IPTG, the SrtA protein will be able to covalently anchor the CelA protein on the surface of the cell. FIG. 2B: SrtA can also covalently anchor a scaffoldin containing a C. thermocellum cohesin domain fused to a CWS that is specifically recognized by SrtA. Strains of B. subtilis have been engineered to co-express B. anthracis SrtA, the scaffoldin protein recognized and displayed by SrtA, and a cellulase (CelA) fused to a C. thermocellum dockerin domain. Upon induction of expression of SrtA with xylose, and IPTG to induce the expression of the scaffoldin protein and the cellulase fusion protein, the scaffoldin protein will be covalently anchored to the cell by SrtA. The cellulase fusion protein will be secreted from the cell, and will specifically bind, in a non-covalent manner, to the cohesin domain within the scaffoldin protein displayed on the surface of the cell. Once the cellulolytic complex assembles on the surface, the enzyme is active and will degrade cellulose.

FIG. 3, panels A-C, provide a schematic illustration of proteins used in this study. Panel A: Schematic of a generalized substrate of the SrtA sortase. SrtA anchored surface proteins contain an N-terminal secretory peptide (SP), a domain that confers function to the protein (surface protein) and a C-terminal cell wall sorting signal (CWS) that contains the LPXTG (SEQ ID NO:14) sorting motif. Panel B: Specific surface proteins that were anchored to the cell wall by SrtA. Each protein contains the SP derived from B. subtilis PhrC which is followed by a hexahistidine (His₆ (SEQ ID NO:16)) or human influenza hemagglutinin (HA) tag. At their C-termini, each protein contains the CWS from the S. aureus Fibronectin Binding Protein B (Fib). CelA is the endoglucanase A from C. thermocellum. Coh is the type I cohesin from the C. thermocellum CipA protein. Scaf contains several domains and has been described previously (see, e.g., Fierobe et al. (2005) J. Biol. Chem. 280: 16325-16334). It contains: the type I cohesin from C. cellulolyticum CipC (Cohc), a Carbohydrate-Binding Module (CBM), a type I cohesin from C. thermocellum CipA (Coht), and a type I cohesin from R. flavefaciens ScaB (Cohf). Panel C: Schematic of the dockerin containing cellulase enzymes that were displayed on the surface of B. subtilis. Proteins purified from E. coli include: CelE-Docc, the C. cellulolyticum exoglucanase CelE fused to its native dockerin (see, e.g., Fierobe et al. (2005) J. Biol. Chem. 280: 16325-16334); CelG-Docf, C. cellulolyticum endoglucanase CelG fused to a type I dockerin from R. flavefaciens ScaA (Id.); and CelA-Doct, the CelA endoglucanase from C. thermocellum fused to the CBM and dockerin modules derived from the C. thermocellum xylanase Xyn10B protein. CelA-Doct-(sec) was used to assemble the cohesin-cellulase complex through co-expression. It is identical to CelA-Doct, except that it contains at its N-terminus the signal peptide from the PhrC protein. Table 6 provides information about each protein.

FIG. 4, panels A-D, show that CelA is successfully displayed on the surface of B. subtilis. Panel A: Immunofluorescence micrographs of B. subtilis strain TDA03 displaying His₆-tagged CelA. Left panel: Cells of strain TDA02 expressing CelA. Middle panel: Cells of strain TDA03 expressing only CelA. Right panel: Cells of strain TDA03 expressing both SrtA and CelA. Cells were probed for the presence of CelA on the surface with mouse anti-His₆ serum and fluorescently stained with goat anti-mouse IgG conjugated to Dylight 488. 4′,6-diamidino-2-phenylindole (DAPI) was used to stain the DNA in order to visualize the cells during microscopy. In images containing larger numbers of cells, a similar display pattern was observed. Panel B: Immunoblot analysis of the cellular localization of CelA in strain TDA03. Lane: 1: purified CelA. 2,6: lysed whole cells. 3,7: lysozyme solubilized cell wall. 4,8: membrane/cytosol. 5,9: precipitated secreted protein. Samples were probed with mouse anti-His6 antibodies that recognize His₆-tagged CelA and visualized using rabbit anti-mouse IgG conjugated to HRP. Lanes 2-5 represent samples in which SrtA was not expressed. Lanes 6-9 represent samples in which SrtA was expressed. Panel C: Diagram showing the CelA-GST protein used to track processing by SrtA. The expected forms of the protein include: P1, the unprocessed full length precursor; P2, the precursor protein after cleavage by the signal peptidase; and M, the mature protein after cleavage of the CWS by SrtA. Panel D: Immunoblots of cell fractions of strain TDA08 expressing CelA-GST and/or SrtA. Top Panel: Precursor proteins P1 and P2 detected in SDS-released cytoplasmic fractions in cells in which SrtA expression has not been induced (SrtA U, left column) or has been induced (SrtA I, right column). Middle Panel: Blot of cell wall extracts that had been digested with mutanolysin. Bottom Panel: detection of SrtA expression in the SDS-treated cytoplasmic and membrane fractions detected using an α-FLAG antibody.

FIG. 5, panels A-C, show that eliminating the WprA protease increases the cellulolytic activity of B. subtilis cells displaying the CelA cellulase. Panel A: Cellulase activity of TDA03 cells during growth. Growth cultures of cells displaying CelA (+SrtA/+CelA) or not displaying CelA (−SrtA/+CelA) were periodically collected, washed and their ability to degrade carboxymethylcellulose determined by measuring the amount of reducing sugars that were released. Panel B: Identical to panel A, except that strain TDA05 was used in which the WprA cell wall associated protease has been genetically deleted. Comparison reveals a 30 fold increase in cell associated activity and increased stability when the protease is deleted. Panel C: Corresponding growth curves of TDA03 and TDA05 as a function of time. Activity profiles were performed in triplicate. The reported error is the standard deviation of these measurements.

FIG. 6, panels A-C, illustrate assembly of a cohesin:cellulase complex on the surface of B. subtilis by either adding purified cellulase or by co-expressing each component. Panel A: Display of the cohesin:cellulase complex after adding purified cellulase enzyme. The panel shows the whole cell activity of cells as a function of time after adding purified cellulase enzyme. Cultures of TDA06 induced to display Coh were grown for varying amounts of time. Purified CelA-Doct was then added, and the ability of washed cells to degrade CMC determined. Little CMC activity is observed in control cultures of TDA06 culture not expressing SrtA or in wild type B. subtilis BAL2238. Panel B: Display of the cohesin:cellulase complex by co-expressing its components. Strain TDA07 was induced to express SrtA, Coh and CelA-Doct-(sec). After varying amounts of time, the cells were washed and the ability to degrade CMC determined. Cultures not expressing SrtA represent controls. Experiments in panels A and B were performed in triplicate and the error reported is the standard deviation. Panel C: Immunoblot of cell wall fractions of strain TDA06 (lanes 1-2) exposed to purified CelA-Doct and strain TDA07 (lanes 3-4) expressing Coh and CelA-Dock-(sec). Lane 1: TDA06 −SrtA/+Coh/+CelA-Doct. Lane 2: TDA06 +SrtA/+Coh/+CelA-Doct. Lane 3: TDA07 +SrtA/+Coh:CelA-Doct-(sec). Lane 4:TDA07 +SrtA/+Coh:CelA-Doct-(sec). Samples were probed using a mouse anti-His₆ antibody to detect His₆-tagged Coh and CelA-Doct and an anti-mouse IgG conjugated to HRP.

FIG. 7, panels A and B, illustrate assembly of a surface displayed minicellulosome that contains three enzymes. Panel A: Immunoblot analysis of the cell wall of cells of strain TDA09 expressing Scaf (lanes 1-4) only, or both SrtA and Scaf (lanes 5-8). Cells were incubated individually with CelA-Doct (lanes 1 and 5), CelE-Docc (lanes 2 and 6), CelG-Docf (lanes 3 and 7) or all three cellulases (lanes 4 and 8). The cell walls were then solubilized and the proteins probed with an anti-His₆ antibody. Panel B: Whole cell activity of cells displaying individual enzymes or a minicellulosome. Cultures of strain TDA09 expressing Scaf and/or SrtA were periodically collected and incubated with purified CelA-Doct, CelE-Docc, or CelG-Docf protein. After washing, activity against HCl-treated amorphous cellulose was determined. Experiments were performed in which only a single enzyme was incubated with the cells (labeled CelA-Doct, CelE-Docc, and CelG-Docf) or in which all three enzymes were used (Minicellulosome (CelA, CelE, CelG). The curve labeled “Sum” is the sum of the enzymatic activities of cells in incubated with only a single type of enzyme. Whole cell cellulase assays were performed in triplicate and the standard deviation of these measurements used to represent the error.

FIG. 8 illustrates a schematic of a new minicellulosome that is capable of degrading biomass. All proteins (scaffolding, sortase and cellulases) are expressed in B. subtilis under an IPTG inducible promoter. C. cellulolyticum endoglucanases Cel48F and Cel5A, and C. cellulolyticum exoglucanase Cel9E possess an N-terminal secretory signal that promotes their secretion. The scaffoldin protein contains three cohesin domains (one from C. cellulolyticum (A), C. thermocellum (D), and one from R. flavefaciens (E). It also contains a cellulose binding module (CB) and N-terminal secretory signal. The scaffoldin also contains a C-terminal cell wall sorting signal recognized by sortase and allows for successful anchoring on the cell surface. On the cell surface the four proteins assemble into a minicellulosome.

FIG. 9 shows that a minicellulosome displaying B. subtilis can degrade biomass and use it as a nutrient. Cells displaying the minicellulosome (TDA10) and those missing the minicellulosome (TDA09) were cultured in minimal medium with biomass as the sole carbon source. The optical densities at 600 nm of the cultures were measured at 24 (white) and 48 (gray) hours. No Glucose and Glucose data are controls and correspond to wild type cells grown in the absence/presence of glucose, respectively.

FIGS. 10A and 10B illustrate growth of cells displaying minicellulosomes or individual cellulases on acid treated corn stover. FIG. 10A: Growth of strain TDA10 (contains Cel5A, Cel48F, Cel9E), strain TDA14 (contains Cel5A and Cel9E), strain TDA12 (contains Cel9E) and strain TDA11 (contains all three enzymes, but lacks scaffoldin). Growth was performed in minimal medium supplemented with acid treated corn stover. Periodically the cell density was measured at 600 nm. FIG. 10B: Total biomass degraded by strain TDA09 (lacks all cellulases) and TDA10. Prior to growth on corn stover, the biomass was weighed. After 72 or 96 hours, the residual biomass was collected, dried and weighed, and the percent consumed determined.

FIG. 11 schematically illustrates an assembled minicellulosome displayed on the surface of a bacterium. The minicellulosome comprises three cellulolytic enzymes joined by three dockerins to three cohesin domains (A, D, and E) on a scaffoldin. The minicellulosome also contains a cellulose binding module (CB).

### DETAILED DESCRIPTION

In various embodiments a method/system is provided for displaying heterologous proteins on the surface of Gram-positive bacteria (e.g., B. subtilis). The system displays proteins using a sortase transpeptidase that covalently anchors proteins to the cell wall of the microbe.

Using this system bacterial strains were developed that can be used to display proteins of interest including, but not limited to cellulolytic proteins on various microorganism, in particular on bacterial strains. In certain embodiments the bacterial strains can be used in the production of biofuels. Specifically, the system can be used to display cellulolytic enzymes (e.g. cellulase enzymes) and multi-enzyme complexes (e.g., cellulosomes, minicellulosomes) on the surface of bacteria (e.g., Gram-positive bacteria such as Bacillus subtilis) through non-covalent interaction with one or more scaffoldin protein(s) that are covalently anchored to the cell wall by a sortase transpeptidase. It is demonstrated herein that the surface displayed protein complexes containing one, or preferably multiple, enzymes are capable of degrading cellulose into its component sugars at high rates. As cellulose is the main component of biomass this suggests that engineering of Gram-positive bacteria such as B. subtilis using this protein display system can create microbes that readily degrade different types of biomass into fermentable sugars.

In various embodiments a single polypeptide fusion can be produced that contained multiple enzymes and a carbohydrate binding module. The sortase will anchor this “multi-enzyme” directly to the cell wall. Alternatively, multiple distinct enzymes could be expressed and each anchored to the cell wall. In both scenarios cell wall attachment of the enzymes will be covalent and typically more stable than other display systems.

These new bacterial strains can be industrially useful in producing biofuels. The Department of Energy (DOE) has mandated by 2020 the increased use of cellulosic ethanol and other biofuels as a transportation fuel. The engineered microbes described herein degrade cellulose more efficiently than previously published engineered organisms. In certain embodiments the microorganism contain three distinct cellulolytic enzymes (e.g., cellulases). Using the same methods, however, engineered organisms containing different cellulolytic enzymes of a multiplicity of such enzymes (e.g., one, two, three, 4, 5, 6, 7, 8, 9, or 10 or more different enzymes) can be produced that possess even greater cellulolytic activity. The modified bacteria described herein also can be used to create a consolidated bioprocessor organism that can directly convert lignocellulosic biomass into bioethanol or other biofuels. In certain embodiments the consolidated bioprocessor can utilize B. subtilis or any other Gram-positive microbe with a conventional cell wall.

In addition to biofuel production, the protein display system described herein has other industrial applications. These include, but are not limited to, creating engineered microbes that are useful in bioremediation, biosensing, and proteomic studies.

We discovered that microorganism growth on biomass is dependent on displayed cellulase activity. Accordingly, the display systems described herein can be used to search for enzymes and enzyme combinations that show improved activity against different types of biomass using growth as a means of selection. It is believed that such a selection method has not been previously possible. One illustrative application is to introduce mutations into the gene encoding cellulase and select for cells with mutated cellulase that grow better on a particular type of biomass. In certain embodiments enzymes would be displayed that are currently being used in industry. Using this selection method they can be improved so that they better degrade a particular type of biomass

Presently, cellulose degradation for biofuel production typically involves rigorous pretreatment of cellulosic materials with chemicals or heat, followed by enzymatic degradation. This is cost inefficient. To overcome this problem several research groups have begun to develop microorganisms that can degrade cellulose. However, it is believed that to date, typical cellulolytic strains have been incapable of degrading cellulose at rates observed for natural cellulolytic organisms.

It was a surprising discovery that the stability, and activity of enzymes displayed on a Gram-positive bacterium can be substantially improved by down regulating or knocking out endogenous proteases in the modified Gram-positive bacterium. In particular, it was demonstrated herein that genetically reducing/eliminating the WprA cell wall protease in B. subtilis (or, by implication, homologues thereof in other Gram-positive bacteria) greatly improved enzyme stability and activity.

Without being bound to a particular theory, it is believed this is the first time that the surface of a bacterium has been engineered to display cell wall attached proteins so as to enable it to degrade cellulose. In general, our work demonstrates the utility of the sortase mediated protein display system for engineering bacterial surfaces for biofuel production. The initial B. subtilis strains we have generated have more potent cellulolytic activity than previously engineered yeast strains.

In various embodiments the systems described herein exploit the use of sortase enzymes to display enzymes (e.g., cellulolytic enzymes). We have shown that sortases are capable of covalently attaching up to ˜300,000 enzymes to the surface of each B. subtilis microbe. We have demonstrated that the high density and stability of the displayed enzymes leads to potent and long lasting cellulolytic activity. As the sortases are prevalent in Gram-positive bacteria the system in principle can be used to engineer the surface of other microbes that could be industrially useful. In particular, thermophilic Geobacillus species can be engineered with these cellulolytic complexes and degrade at high temperatures which can further enhance the activity of the cellulolytic enzymes within the complex.

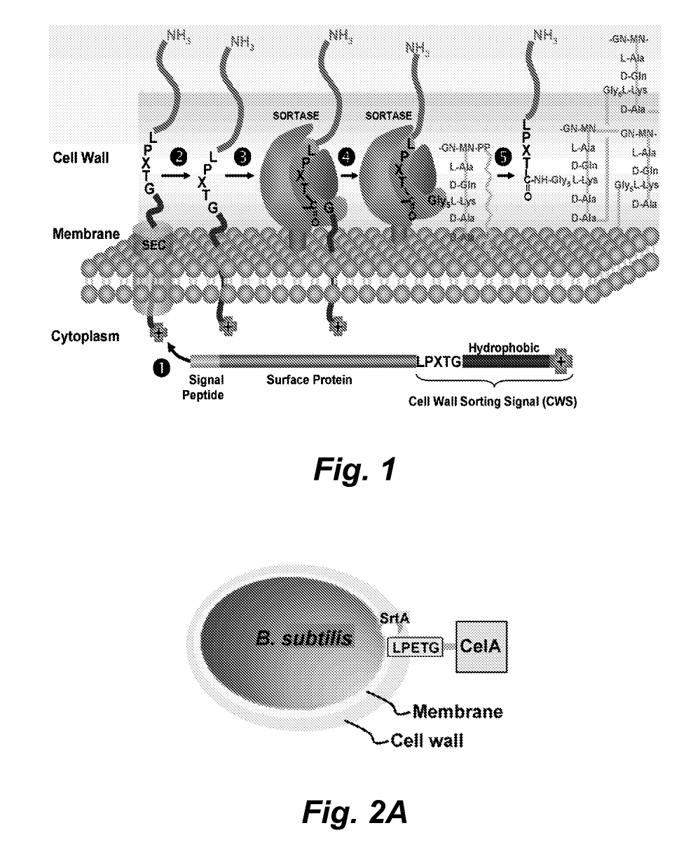

In various illustrative embodiments, the display systems works by co-expressing a sortase enzyme (e.g., the B. anthracis sortase A enzyme) with a protein substrate that it covalently anchors to the cell wall. The sortase A enzyme used in the illustrative embodiments shown herein is a transpeptidase that recognizes a certain amino acid motif (leucine, proline, any amino acid, threonine, and glycine (LPXTG, SEQ ID NO:14), also known as a cell wall sorting signal, within the protein that is to be displayed on the surface of the cell. Once the transpeptidase recognizes this motif it covalently attaches the protein by joining the threonine carbonyl group within the motif to the diaminopimelic acid (dap) motif within the cell wall (see, e.g., FIG. 1).

Without being bound to a particular theory, it is believed that the sortase (e.g., sortase A) catalyzes the transpeptidation reaction by first cleaving the protein substrate at the cell wall sorting signal. The resulting acyl enzyme intermediates between sortases and their substrates are then resolved by the nucleophilic attack of amino groups, typically provided by the cell wall cross bridges of peptidoglycan precursors. The product of the sortase reaction, a surface protein linked to peptidoglycan, is then incorporated into the envelope and displayed on the microbial surface.

Thus, after the transpeptidation occurs, the protein of interest is covalently anchored to the peptidoglycan and successfully displayed on the surface of the cell. In various embodiments the protein(s) to be displayed will typically carry two topogenic sequences, N-terminal signal peptides and C-terminal sorting signals. In various embodiments the cell wall sorting signals span approximately 30 to 40 residues and comprise a short pentapeptide motif followed by a stretch of hydrophobic side chains and finally a mostly positively charged tail at the C-terminal end of the polypeptide. The sortase (e.g., Sortase A) is a central factor in the so-called “sorting pathway.” This pathway begins with the protein to be displayed precursor in the cytoplasm. The N-terminal signal peptide then directs the precursor to the membrane for translocation. Once the signal peptide has been cleaved and the polypeptide is moved across the plasma membrane, the cell wall sorting signal functions to retain the polypeptide within the secretory pathway. Membrane-anchored sortases cleave sorting signals at their pentapeptide motif and promote anchoring to the cell wall.

Accordingly, in various embodiments, the protein to be displayed also contains a secretory signal peptide that directs its secretion from the cell. In one illustrative embodiment, to increase the stability of the displayed proteins the WprA cell protease was genetically eliminated.

In one approach to display multiple proteins on the surface of the cell, a scaffoldin protein is covalently displayed on the surface of the cell using the sortase transpeptidase, e.g., as described above. The scaffoldin protein contains one or more cohesin domains (e.g., a type I cohesin domain from Clostridium thermocellum). The cohesin domain(s) then bind to any protein that contains a matching dockerin domain (e.g., a type I dockerin domain from C. thermocellum).

In the model system illustrated in the Examples, an endoglucanase from C. thermocellum was fused to a type I dockerin domain. This fusion protein also contained an optional cellulose binding domain (CBD) that facilitates interactions with the cellulose substrate. The fusion protein was then co-expressed with the sortase enzyme and its cognate scaffoldin containing the appropriate cohesin domain. The dockerin-fused endoglucanase was then shown to be successfully displayed on the surface via interactions with the scaffoldin. The displayed endoglucanase had potent enzymatic activity.

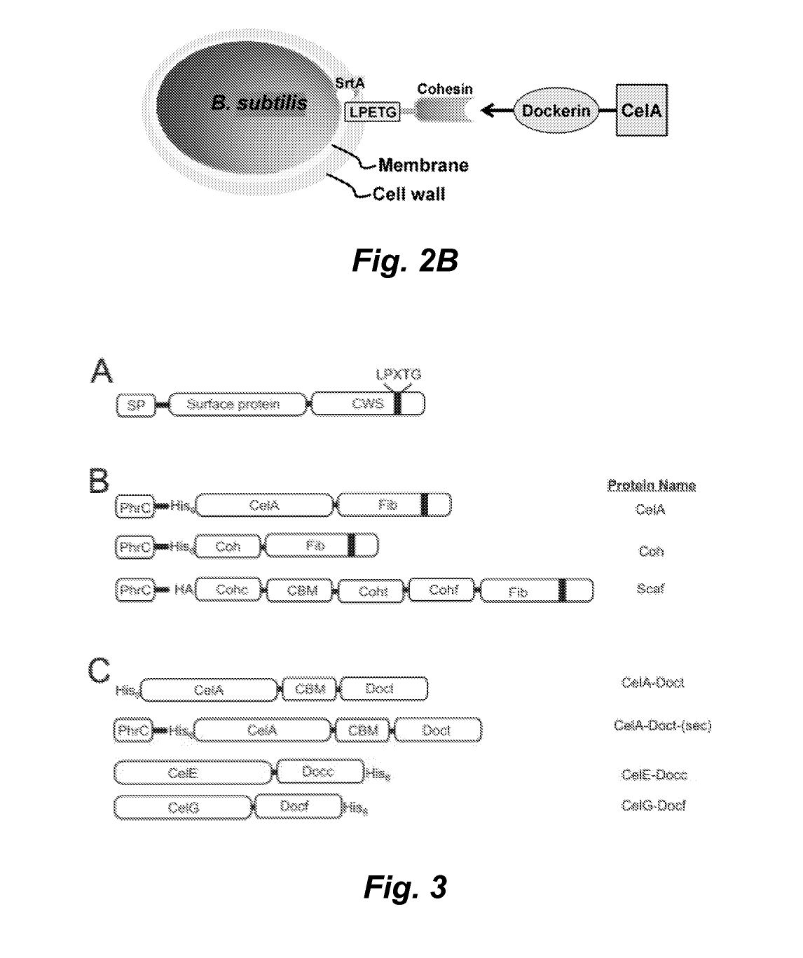

We have also shown that the same strategy can be used to incorporate three or more distinct cellulolytic enzymes onto the scaffoldin. In one illustrative embodiment, this was accomplished by creating a scaffoldin that contained three cohesin domains that each had unique binding specificities for different types of dockerin domains. Three different cellulase-dockerin fusion proteins were specifically incorporated into the scaffoldin by binding via their dockerin domain to a unique cognate cohesin domain within the scaffoldin (see, e.g., FIGS. 1 and 2) schematically illustrating the sortase transpeptidase reaction and how it was used in the exemplified system.

In certain embodiments the system described herein can be used to generate cellulolytic strains of B. subtilis, or other Gram-positive microorganisms. The bacteria can be applied to biomass to degrade it into its component sugars. The endoglucanase displayed on the bacterial surface would degrade the cellulose into its component sugars. The sugars produced can then be used as feedstock for fermentation reactions that will produce biofuels.

It was demonstrated that the surface displayed cellulolytic activity is stable for several days. Therefore, dormant (non-growing cells) can be applied to the biomass to degrade it. Degradation could occur over several days and the released sugars could be harvested. Alternatively, in certain embodiments, the Gram-positive bacterial cells (e.g., B. subtilis cells) can be co-cultured with biomass. As compared to current methods to degrade biomass this method could be advantageous. This is because it can eliminate the requirement of applying purified enzymes to the biomass solution. In principle, it can also eliminate the need to pretreat the biomass. Importantly, the system described herein can be used to construct B. subtilis, or other Gram-positive bacterial, cells containing several different types of cellulolytic (or other) enzymes. These microbes have increased degradative power and can be tailored to degrade different types of biomass. The systems described herein can also be ported to other Gram-positive microbes that may have desirable industrial properties. Some of these organisms are already capable of producing biofuels and include, but are not limited to thermophilic Geobacillus (e.g. Geobacillus thermoglucosidasius) or Clostridia (e.g. C. thermocellum) species. In certain embodiments, to eliminate import of degraded cells into growing microbes it is possible to eliminate (knockout) genes that encode for sugar importers.

In certain embodiments the system described herein can be used to generate B. subtilis (or other Gram-positive bacteria) that display a cellulolytic enzyme (e.g., endoglucanase). Unlike the native organism, this B. subtilis strain can use cellulose as a nutrient source. This creates the opportunity to engineer B. subtilis (or other Gram-positive bacteria) so as to make a consolidated bioprocessor capable of converting biomass into biofuels. To accomplish this goal additional genes can be introduced into the organism so to establish metabolic pathways that convert glucose (the product of cellulose degradation) into bioethanol or other biofuels. Similar metabolic pathways to make biofuels have been introduced into E. coli, so that, using the teachings provided herein, it is feasible that similar pathways can be introduced into B. subtilis or other Gram-positive bacteria.

### Cell Wall Sorting Signal

As explained herein, in various embodiments, a sortase transpeptidase (e.g., Sortase A or analogues, homologues, or orthologues thereof) is exploited to couple a protein (e.g., a protein comprising an enzyme (e.g. cellulolytic enzyme) and/or one or more cohesion domain to the surface (e.g., cell wall) of a Gram-positive microorganism. To facilitate this, the peptide is provided with a cell wall sorting signal sequence that is recognized by the sortase transpeptidase.

The examples provided herein use a C-terminal portion of the Staphylococcus aureus Fibronectin Binding Protein B, which contains a 123 amino acid spacer segment and the cell wall sorting signal (CWS). This S. aureus CWS sequence is identical to CWS found in many B. anthracis surface proteins that are anchored to the cell wall of B. anthracis by SrtA

Typically cell wall sorting signals comprise an LPXTG (SEQ ID NO:14) motif (where X is any amino acid), a C-terminal hydrophobic domain and a charged tail. Homologous sequences are found in many surface proteins of Gram-positive bacteria (see, e.g., Schneewind et al. (1993) EMBO J., 12(12): 4803-4811, which describes a number of cell wall sorting signals, illustrated below in Table 1).

TABLE 1
Illustrative cell wall sorting signals in surface
proteins of Gram-positive bacteria.

| | | SEQ | |
|---|---|---|---|
| | | ID | Bacterial |
| Protein | Cell Wall Sorting Signal | NO: | Species |
| SPA | LPETGEENPFIGTTVFGGLSLALGAALLAGRRREL | 2 | S. aureus |
| FNBP | LPETGGEESTNKGMLFGGLFSILGLALLRRNKKNH | 3 | S. aureus |
| | KA | | |
| SPAA | LPATGDSSNAYLPLLGLVSLTAGFSLLGLRRKQD | 4 | S. sobrinus |
| PRGB | LPKTGEKQNVLLTVVGSLAAMLGLAGLGFKRRKET | 5 | E. faecalis |
| | K | | |
| TEE | LPSTGSIGTYLFKAIGSAAMIGAIGIYIVKRRKA | 6 | S. pyogenes |
| INLA | LPTTGDSDNALYLLLGLLAVGTAMALTKKARASK | 7 | L. |
| | | | monocytogenes |
| FIM. | LPLTGANGVIFLTIAGALLVAGGAVVAYANKRRHV | 8 | A. viscosus |
| | AKH | | |
| BAC | LPYTGVASNLVLEIMGLLGLIGTSFIAMKRRKS | 9 | S. agalactiae |
| CNA | LPKTGMKIITSWITWVFIGILGLYLILRKRFNS | 10 | S. aureus |
| WAP | LPSTGEQAGLLLTTVGLVIVAVAGVYFYRTRR | 11 | S. mutans |
| EMM | LPSTGETANPFFTAAALTVMATAGVAAVVKRKEEN | 12 | S. pyogenes |

These cell wall sorting signals are intended to be illustrative and not limiting. Using the teachings provided herein, numerous other cell wall sorting signals can be incorporated in the expression/display systems described herein.

While in certain embodiments, cell wall sorting signals comprising the LPT are preferred, they need not be limited to this motif. Based on homology sortases thus far identified are typically grouped into four or five subgroups or classes (see, Table 2). Each subgroup, in addition to distinctions in sequence, can be distinguished from one another based on membrane topology, genome position, and preference for substrates with specific amino acids within the cell wall sorting signal pentapeptide motif (Comfort and Clubb (2004) Infect. Immun., 72: 2710-2722; Dramsi et al. (2005) Res. Microbiol. 156: 289-297). As indicated above, the prototypical sortase is sortase A, first identified in S. aureus. Sortase A appears to anchor a large number and broad range of surface proteins. The sortase A subgroup of enzymes also seems to share a preference for the LPXTG (SEQ ID NO:14) cell wall sorting signal motif. The second subgroup of enzymes, sortase B, along with its substrate (IsdC in S. aureus), is encoded in an iron transport operon involved in heme-iron uptake. Enzymes belonging to the sortase B subgroup contain three amino acid segments not found in sortase A and recognize substrates containing an NPQTN (SEQ ID NO:17) motif rather than the canonical LPXTG (SEQ ID NO:14). The third class, designated sortase C or subfamily 3, contains a C-terminal hydrophobic domain (Id.). Subfamily 3 enzymes also share a preference for substrates containing the LPXTG cell wall sorting signal motif, often followed by a second G residue (i.e., LPXTGG, (SEQ ID NO:18). A fourth subgroup can be defined after alignment of sortase sequences. It has been noted as the sortase D subgroup (Dramsi et al. (2005) Res. Microbiol. 156: 289-297) or subfamilies 4 and 5, as sortases in this subgroup can be distinguished based on the cell wall sorting signals of their associated substrates (Comfort and Clubb (2004) Infect. Immun., 72: 2710-2722). Sortases belonging to subfamily 4 are predicted to anchor proteins bearing the unique LPXTA(ST) (SEQ ID NO:19) motif (Id.). An alanine residue in the last position of the substrate motif suggests that the subfamily 4 enzymes fulfill a nonredundant role within the cell (Id.). Many high-G/C bacteria contain sortases belonging to subfamily 5, and most do not harbor sortase A. This subgroup of sortase enzymes shares substrate specificity for proteins containing an LAXTG (SEQ ID NO:20) motif (Id.).

TABLE 2
Sortase classifications.

| Sortase | | Membrane | |
|---|---|---|---|
| class | Cleavage | anchor | |
| (subfamily)a | site^{b} | domain^{c} | Bacterial taxa^{d} |
| A (1) | LPkT-Ge* | N terminus | Bacillus, Listeria, |
| | | | Staphylococcus, |
| | | | Enterococcus, |
| | | | Lactobacillaceae, |
| | | | Streptococcaceae |
| B (2) | NPqt-nd* | N terminus | Bacillus, Listeria, |
| | | | Staphylococcus, |
| | | | Streptococcaceae, |
| | | | Clostridia |
| C (3) | 1PkT-GG | C terminus | Actinobacteria, |
| | | | Bacillus, Enterococcus, |
| | | | Leuconostocaceae, |
| | | | Streptococcaceae, |
| | | | Clostridia |
| D (4) | LPnT-At | N terminus | Bacillus |
| D (5) | LAeT-Ga | N terminus | Actinobacteria |

aSortase subfamily and class assignments are based on sequence, membrane topology, genomic positioning, and preference for specific amino acids within the cell wall sorting signal pentapeptide motif region of their cognate substrates.
^{b}Cell wall sorting signal pentapeptide motif. Uppercase letters represent amino acids that are absolutely conserved. Asterisks indicate that the cleavage site has been verified experimentally.

Accordingly in various embodiments, display systems that utilize any of these cell wall sorting sequences are contemplated for use in the methods and constructs described herein.

### Cohesin and Dockerin Proteins/Protein Domains.

As described herein, in various embodiments, the display system(s) utilize one or more proteins (e.g., scaffoldins) comprising one or more cohesin domains (e.g., cohesin I domains) that interact with dockerin domains to anchor and/or organize one or more enzymes on the surface of the Gram-positive bacterium.

In various embodiments the systems contemplated herein can comprise one or more dockerin domains selected from the group consisting of a dockerin I domain, a dockerin II domain, and a dockerin III domains. Correspondingly, in various embodiments the systems contemplated herein can comprise one or more cohesin domains selected from the group consisting of a cohesin I domain, a cohesin II domain, and a cohesin III domains that binds to its corresponding dockerin sequence. In certain embodiments the dockerin and/or cohesin domains comprise a domain derived from Clostridium thermocellum.

The sequences of cohesins and dockerins are well known to those of skill in the art (see, e.g., Ding et al. (2003) Genet. Eng. (NY) 25: 209-225 for cellulosome cohesins and dockerins, and. Peer et al. (2009) FEMS Microbiol Lett. 291(1): 1-16 for non-cellulosome cohesins and dockerins). In various embodiments specific cohesin-dockerin pairs are chosen so as to enable specific complexes to form on the cell surface even if multiple enzymes are present.

While the display system described herein is exemplified using cohesin-dockerin pairs, it will be recognized that, in certain embodiments, any protein-protein interaction pair can be used as long as one member of the pair becomes covalently attached to the cell wall and the other is fused to the cellulolytic enzyme(s) so as to enable enzyme complex formation on the cell surface.

### Cellulolytic Enzymes and Minicellulosomes.

In various embodiments Gram-positive bacteria are engineered using the methods described herein to display one or more enzymes. In certain embodiments the enzymes are cellulolytic enzymes and/or other enzymes useful in the synthesis of biofuels from lignocellulosic biomass. In various embodiments it will be recognized that the “cellulases” can include, but are not limited to, the cellobiohydrolases, e.g., cellobiohydrolase I and cellobiohydrolase II, as well as the endoglucanases. In various embodiments “cellulolytic enzymes” include, but are not limited to, cellobiohydrolases, e.g. cellobiohydrolase I and cellobiohydrolase II, as well as endoglucanases and beta-glucosidases.

In various embodiments the digestion of cellulose and hemicellulose is facilitated by the use of several types of enzymes acting cooperatively. In certain embodiments at least three categories of enzymes are utilized to convert cellulose into fermentable sugars: endoglucanases that cut the cellulose chains at random; cellobiohydrolases that cleave cellobiosyl units from the cellulose chain ends and beta-glucosidases that convert cellobiose and soluble cellodextrins into glucose. Among these three categories of enzymes involved in the biodegradation of cellulose, cellobiohydrolases are useful for the degradation of native crystalline cellulose. Cellobiohydrolase I, also referred to as a cellulose 1,4-beta-cellobiosidase or an exoglucanase, exo-cellobiohydrolase or 1,4-beta-cellobiohydrolase catalyzes the hydrolysis of 1,4-beta-D-glucosidic linkages in cellulose and cellotetraose, by the release of cellobiose from the non-reducing ends of the chains. Cellobiohydrolase II activity is identical, except that cellobiohydrolase II attacks from the reducing ends of the chains.

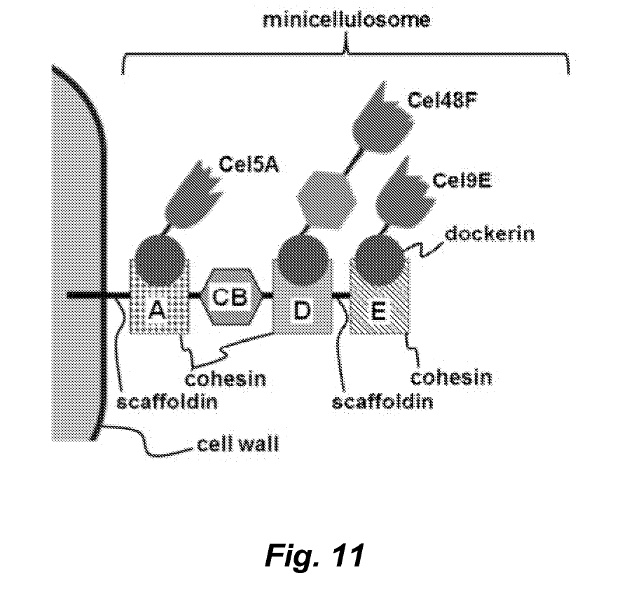

In various embodiments the cellulolytic enzymes are organized into a cellulosome or minicellulosome (see, e.g., FIGS. 2B and 11). Cellulosome complexes are multi-enzyme complexes that can be designed for efficient degradation of plant cell wall polysaccharides, notably cellulose. Cellulosomes typically comprises a multifunction integrating scaffold (called scaffoldin), responsible for organizing the various cellulolytic subunits (e.g., the enzymes) into the complex. The scaffolidin comprises one or more cohesin domains. Enzymes attached to dockerins are organized on the scaffoldin by specific interactions between cohesins and dockerins that specifically or preferentially bind to particular cohesins. In certain embodiments attachment of the cellulosome to its substrate is mediated by a scaffoldin-borne cellulose-binding module (CBM) that can comprises part of the scaffoldin subunit.

The displayed cellulosomes can be simple cellulosome systems containing a single scaffoldin or complex cellulosome systems that exhibit multiple types of interacting scaffoldins. In various embodiments each scaffoldin can contain one, two, three, four, five, six, seven, eight, nine, or 10 or more cohesin domains. The arrangement of the modules on the scaffoldin subunit and the specificity of the cohesin(s) and/or dockerin for their modular counterpart determine the overall architecture of the cellulosome. Several different types of scaffoldins have been described and are useful in the construction of minicellulosomes according to the methods described herein. The primary scaffoldins incorporate the various dockerin-bearing subunits directly into the cellulosome complex, adaptor scaffoldins increase the repertoire or number of components into the complex, and the anchoring scaffoldins attach the complex to the bacterial cell surface.

Scaffoldins are well known to those of skill in the art and can readily be identified with a simple GenBank search for the term “scaffoldin”.

In certain embodiments the cellulolytic enzymes comprising the cellulosome or individually displayed on the surface of the bacteria comprise one or more enzymes collected from the group consisting of an exoglucanase, an endoglucanase, a glycosyl hydrolase, a cellulase, a hemicellulase, a xylanase, a cellobiohydrolase, a beta-glucosidase, a mannanse, a xylosidase (e.g., a β-xylosidase), an arabinofuranosidase, and/or a glucose oxidase. Illustrative enzymes suitable for display using the systems described herein are shown in Table 3.

TABLE 3
Illustrative enzymes suitable for display on Gram-
positive bacteria using the methods described herein.

| | Genbank |
|---|---|
| Enzyme | Accession No |
| Endoglucanases: | |
| Clostridium thermocellum endoglucanse CelG | 69390 |
| Clostridium thermocellum endoglucanse CelD | X04584 |
| Clostridium thermocellum endoglucanse CelQ | AB047845 |
| Clostridium thermocellum endoglucanse CelR | AJ585346 |
| Clostridium thermocellum endoglucanse CelN | AJ275974 |
| Exoglucanases: | |
| Clostridium thermocellum exoglucanase CelS | L06942 |
| Cellobiohydrolases: | |
| Clostridium thermocellum cellobiohydrolase CbhA | X80993 |
| Clostridium thermocellum cellobiohydrolase CelK | AF039030 |
| Clostridium thermocellum cellobiohydrolase CelO | AJ275975 |
| Xylanases: | |
| Clostridium thermocellum xylanase XynD | AJ585345 |
| Clostridium thermocellum xylanase XynC | D84188 |
| Clostridium thermocellum xylanase XynA | AB010958 |
| Hemicellulases: | |
| Clostridium thermocellum lichenase LicB | X63355 |
| Clostridium thermocellum chitinase ChiA | CAB52403 |
| Clostridium thermocellum mannase ManA | CAD12659 |

In addition, a large number of other suitable enzymes are described in U.S. Patent Publication 2010/0189706 which is incorporated herein by reference for any one or more of the cellulolytic enzymes described herein. Cellulosomes are also described by Fontes and Gilbert (2010) Annu. Rev. Biochem., 79: 655-681.

In certain embodiments the cellulosome that is to be displayed can be engineered based upon the cellulosic material to be metabolized. For example, different cellulases and other enzymes may be engineered into a cellulosome pathway depending upon the sources of substrate. Illustrative substrate sources include, but are not limited to, alfalfa, corn stover, crop residues, debarking waste, forage grasses, forest residues, municipal solid waste, paper mill residue, pomace, sawdust, spent grains, spent hops, switchgrass, and wood chips. Some substrate sources can have a larger percentage of cellulose compared to other source, which may have a larger percentage of hemicellulose.

A hemicellulose substrate typically comprises short, branched chains of sugars and can comprise a polymer of five different sugars. Hemicellulose comprises five-carbon sugars (e.g., D-xylose and L-arabinose) and six-carbon sugars (e.g., D-galactose, D-glucose, and D-mannose) and uronic acid. The sugars are typically substituted with acetic acid. Hemicellulose is relatively easy to hydrolyze to its constituent sugars. When hydrolyzed, the hemicellulose produces xylose (a five-carbon sugar) or six-carbon sugars from hardwoods or softwoods, respectively.

Proteins or polypeptides having the ability to convert the hemicellulose components into carbon sources that can be used as a substrate for biofuel production includes, for example, cellobiohydrolases (Accessions: AAC06139, AAR87745, EC 3.2.1.91, 3.2.1.150), cellulases (E.C. 3.2.1.58, 3.2.1.4, Accessions: BAA12070, BAB64431); chitinases (E.C. 3.2.1.14, 3.2.1.17, 3.2.1.-, 3.2.1.91, 3.2.1.8, Accessions: CAA93150, CAD12659), various endoglucanases (E.C. 3.2.1.4, Accessions: BAA92430, AAG45162, P04955, AAD39739), exoglucanases (E.C. 3.2.1.91, Accessions: AAA23226), lichenases (E.C. 3.2.1.73, Accessions: P29716), mannanases (E.C. 3.2.1.4, 3.2.1.-, Accessions: CAB52403), pectate lyases (E.C. 4.2.2.2, Accessions: AAG59609), xylanase (E.C. 3.2.1.136, 3.2.1.156, 3.2.1.8, Accessions: BAA33543, CAA31 109) and silase (E.C. 3.2.2.-, 2.7.7.7, Accessions: CQ800975).

Cellulases are a class of enzymes produced chiefly by fungi, bacteria, and protozoans that catalyze the hydrolysis of cellulose. However, there are also cellulases produced by other types of organisms such as plants and animals. Several different kinds of cellulases are known, which differ structurally and mechanistically. The EC number for cellulase enzymes is E.C.3.2.1.4. Assays for testing cellulase activity are known in the art.

Polypeptides having xylanase activity are also useful in synthetic cellulosomes. Xylanase is the name given to a class of enzymes which degrade the linear polysaccharide beta-1,4-xylan into xylose, thus breaking down hemicellulose. The EC number for xylanase enzymes is E.C. 3.2.1.136, 3.2.1.156, 3.2.1.8. Assays for testing xylanase activity are known in the art.

In certain embodiments the minicellulosome comprises at least two different cellulolytic (or other degredative) enzymes. In certain embodiments the two enzymes comprise an enzyme pair selected from the group consisting of endocellulase/endocellulase, exocellulase/endocellulase, beta-glucosidase (cellobiase)/endocellulase, oxidative cellulase/endocellulase, xylanase/endocellulase, hemicellulase/endocellulase, lichenase/endocellulase, chitenase/endocellulase, xylanase/endocellulase, cellulose phosphorylase/endocellulase, endocellulase/exocellulase, exocellulase/exocellulase, beta-glucosidase (cellobiase)/exocellulase, oxidative cellulase/exocellulase, xylanase/exocellulase, hemicellulase/exocellulase, lichenase/exocellulase, chitenase/exocellulase, xylanase/exocellulase, cellulose phosphorylase/exocellulase, endocellulase/beta-glucosidase, exocellulase/beta-glucosidase, beta-glucosidase (cellobiase)/beta-glucosidase, oxidative cellulase/beta-glucosidase, xylanase/beta-glucosidase, hemicellulase/beta-glucosidase, lichenase/beta-glucosidase, chitenase/beta-glucosidase, xylanase/beta-glucosidase, cellulose phosphorylase/beta-glucosidase, endocellulase/oxidative cellulase, exocellulase/oxidative cellulase, beta-glucosidase (cellobiase)/oxidative cellulase, oxidative cellulase/oxidative cellulase, xylanase/oxidative cellulase, hemicellulase/oxidative cellulase, lichenase/oxidative cellulase, chitenase/oxidative cellulase, xylanase/oxidative cellulase, cellulose phosphorylase/oxidative cellulase, endocellulase/xylanase, exocellulase/xylanase, beta-glucosidase (cellobiase)/xylanase, oxidative cellulase/xylanase, xylanase/xylanase, hemicellulase/xylanase, lichenase/xylanase, chitenase/xylanase, xylanase/xylanase, cellulose phosphorylase/xylanase, endocellulase/hemicellulase, exocellulase/hemicellulase, beta-glucosidase (cellobiase)/hemicellulase, oxidative cellulase/hemicellulase, xylanase/hemicellulase, hemicellulase/hemicellulase, lichenase/hemicellulase, chitenase/hemicellulase, xylanase/hemicellulase, cellulose phosphorylase/hemicellulase, endocellulase/lichenase, exocellulase/lichenase, beta-glucosidase (cellobiase)/lichenase, oxidative cellulase/lichenase, xylanase/lichenase, hemicellulase/lichenase, lichenase/lichenase, chitenase/lichenase, xylanase/lichenase, cellulose phosphorylase/lichenase, endocellulase/chitenase, exocellulase/chitenase, beta-glucosidase (cellobiase)/chitenase, oxidative cellulase/chitenase, xylanase/chitenase, hemicellulase/chitenase, lichenase/chitenase, chitenase/chitenase, xylanase/chitenase, cellulose phosphorylase/chitenase, endocellulase/xylanase, exocellulase/xylanase, beta-glucosidase (cellobiase)/xylanase, oxidative cellulase/xylanase, xylanase/xylanase, hemicellulase/xylanase, lichenase/xylanase, chitenase/xylanase, xylanase/xylanase, cellulose phosphorylase/xylanase, endocellulase/cellulose phosphorylase, exocellulase/cellulose phosphorylase, beta-glucosidase (cellobiase)/cellulose phosphorylase, oxidative cellulase/cellulose phosphorylase, xylanase/cellulose phosphorylase, hemicellulase/cellulose phosphorylase, lichenase/cellulose phosphorylase, chitenase/cellulose phosphorylase, xylanase/cellulose phosphorylase, and cellulose phosphorylase/cellulose phosphorylase.

In certain embodiments the minicellulosome comprises at least three different cellulolytic (or other degredative) enzymes. In certain embodiments the three different enzymes comprise an enzyme pair selected from the group listed above, combined with one enzyme selected from the group consisting of an endocellulase, an exocellulase, a beta-glucosidase (cellobiase), an oxidative cellulase, a xylanase, a hemicellulase, a lichenase, a chitenase, a xylanase, and a cellulose phosphorylase.

It will be recognized that the enzymes, and enzyme combinations, identified above are intended to be illustrative and not limiting. Using the teachings provided herein, the display of numerous other enzymes will be available to one of skill in the art.

### Carbohydrate Binding Domain/Module (CBD/CBM)

In various embodiments, to facilitate interaction of displayed enzyme(s) with their substrate (e.g., cellulose) the displayed protein comprises a substrate binding domain (e.g., a carbohydrate binding domain). Suitable substrate binding domains include, but are not limited to, carbohydrate binding domains, cellulose binding domains, cellulose binding modules, or other binding domains.

The amino acid sequence of cellulose binding peptides and/or binding domains are well known to those of skill in the art. Carbohydrate binding peptides include peptides e.g., proteins and domains (portions) thereof, that are capable of, binding to a plant derived cellulosic (e.g., lignocellulosic) material. Carbohydrate binding peptides include, for example, peptides screened for their cellulose binding activity out of a library, as well as naturally occurring cellulose binding peptides or peptide domains.

The carbohydrate binding domain can include any amino acid sequence expressible in plants which binds to a cellulose polymer. For example, the cellulose binding domain or protein can be derived from a cellulase, a binding domain of a cellulose binding protein or a protein screened for, and isolated from, a peptide library, or a protein designed and engineered to be capable of binding to cellulose or to saccharide units thereof. The cellulose binding domain or protein can be naturally occurring or synthetic. Suitable polysaccharidases from which a carbohydrate binding domain can be obtained includes, but is not limited to a β-1,4-glucanase. In certain embodiments, a cellulose binding domain or protein from a cellulase or scaffoldin is used.

Carbohydrate binding domains/modules are well known to those of skill in the art (see, e.g., Tomme et al. (1995) in Enzymatic Degradation of Insoluble Polysaccharides (Saddler and Penner, eds.), Cellulose-binding domains: classification and properties. pp. 142-163, American Chemical Society, Washington). Cellulose binding domains are also described in U.S. Pat. No. 5,837,814 and in U.S. Patent publication 2011/0005697 which are incorporated herein by reference for the cellulose binding domains described therein. In particular, U.S. Patent Publication No: 2011/0005697 identifies proteins containing putative β-1,3-glucan-binding domains (see, e.g., Table 1 therein, Table 4 below); proteins containing Streptococcal glucan-binding repeats (Cp1 superfamily) (see e.g., Table 2 therein, Table 5 below), and the like.

TABLE 4
Proteins containing putative β-1,3 glucan binding domains.

| Source (strain) | Protein | Accession No. | Ref |
|---|---|---|---|
| Type I: | | | |
| B. circulans (WL-12) | GLCA1 | P23903/M34503/JQ0420 | 1 |
| B. circulans (IAM 1165) | BglH | JN0772/D17519/267033 | 2 |
| Type II: | | | |
| Actinomadura sp. (FC7) | XynII | U08894 | 3 |
| Arthrobacter sp. | GLCI | D23668 | 10 |
| (YCWD3) | | | |
| O. xanthineolytica | GLC | P22222/M60826/A39094 | 4 |
| R. faecitabidus | RPI | Q05308/A45053/D10753 | 5, 6 |
| (YLM-50) | | | |
| R. communis | Ricin | A12892 | 7 |
| S. ividans (1326) | XlnA | P26514/M64551/JS07986 | 8 |
| R. tridentatus | FactorGa | D16622 | 9 |

1 Yahata et al. Gene, 86: 113-117
2 Yamamoto et al.(1993) Biosci. Biotechnol. Biochem., 57: 1518-1525.
3 Harpin et al. (1994) EMBL Data Library.
4 Shen et al. (1991) J. Biol. Chem., 266: 1058-1063
5 Shimoi et al. (1992) J. Biol. Chem., 267: 25189-25195.
6 Shimoi et al. (1992) J. Biochem., 110: 608-613
7 Horn et al. (1989) Patent A12892
8 Shareck et al. (1991) Gene, 269: 1370-1374.
9 Seki et al. (1994) J. Biol. Chem., 269: 1370-1374
10 Watanabe et al. (1993) EMBL Data Library.

TABLE 5
Illustrative proteins containing Streptococcal
glucan-binding repeats.

| Source | Protein | Accession No. | Ref. |
|---|---|---|---|
| S. downei (sobrinus) | GTF-I | D13858 | 1 |
| S. downei (sobrinus) | GTF-I | P11001/M17391 | 2 |
| S. downei (sobrinus) | GTF-S | P29336/M30943/A41483 | 3 |
| S. downei (sobrinus) | GTF-I | P27470/D90216/A38175 | 4 |
| S. downei (sobrinus) | DEI | L34406 | 5 |
| S. mutans (Ingbritt) | GBP | M30945/A37184 | 6 |
| S. mutans (GS-5) | GTF-B | A33128 | 7 |
| S. mutans (GS-5) | GTF-B | P08987/M17361/B33135 | 8 |
| S. mutans | GTF-B^{3′-ORF} | P05427/C33135 | 8 |
| S. mutans (GS-5) | GTF-C | P13470/M17361/M22054 | 9 |
| S. mutans (GS-5) | GTF-C | Not available | 10 |
| S. mutans (GS-5) | GTF-D | M29296/A45866 | 11 |
| S. salivarius | GTF-J | A44811/S22726/S28809/ | 12 |
| | | Z11873/M64111 | |
| S. salivarious | GTF-K | S22737/S22727/Z11872 | 13 |
| S. salivarious | GTF-L | L35495 | 14 |
| (ATCC25975) | | | |
| S. salivarious | GTF-M | L35928 | 14 |
| (ATCC25975) | | | |
| S. pneumoniae R6 | LytA | P06653/A25634/M13812 | 15 |
| S. pneumoniae | PspA | A41971/M74122 | 16 |
| Phage HB-3 | HBL | P06653/A25634/M13812 | 17 |
| Phage Cp-1 | CPL-1 | P15057/J03586/A31086 | 18 |
| Phage CP-9 | CPL-9 | P19386/M34780/JQ0438 | 19 |
| Phage EJ-1 | EJL | A42936 | 20 |
| C. difficile | ToxA | P16164/A37052/M30307/ | 21 |
| (VPI 10463) | | X51797/S08638 | |
| C. difficile | ToxA | A60991/X17194 | 22 |
| (BARTS W1) | | | |
| C. difficile | ToxB | P18177/X53138/X6098/ | 23, 24 |
| (VPI 10463) | | S10317 | |
| C. difficile | ToxB | X44271/Z23277 | 25, 26 |
| (1470) | | | |
| C. novyi | α-toxin | S44272/Z23280 | 27 |
| C. novyi | α-toxin | Z48636 | 28 |
| C. acetobutylicum | CspA | 549225/Z37723 | 29 |
| (NCIB8052) | | | |
| C. acetobutylicum | CspB | Z50008 | 30 |
| (NCIB8052) | | | |
| C. acetobutylicum | CspC | Z50033 | 30 |
| (NCIB8052) | | | |
| C. acetobutylicum | CspD | Z50009 | 30 |
| (NCIB8052) | | | |

References:
1 Sato et al. (1993) DNA sequence 4: 19-27
2 Ferreti et al. (1987) J. Bacteriol., 169: 4271-4278
3 Gilmore et al. (1990) J. Infect. Immun., 58: 2452-2458.
4 Abo et al. (1991) J. Bacteriol., 173: 989-996.
5 Sun et al. (1994) J. Bacteriol., 176: 7213-7222.
6 Banas et al. (1990) J. Infect. Immun., 58: 667-673.
7 Shiroza et al. (1990) Protein Sequence Database.
8 Shiroza et al. 91987) J. Bacteriol., 169: 4263-4270.
9 Ueda et al. (1988) Gene, 69: 101-109.
10 Russel (1990) Arch. Oral Biol., 35: 53-58.
11 Honda et al. (1990) J. Gen. Microbiol., 136: 2099-2105
12 Giffard et al. (1991) J. Gen. Microbiol., 137: 2577-2593.
13 Jacques (1992) EMBL Data Library
14 Simpson et al. (1995) J. Infect. Immun., 63: 609-621
15 Garcia et al. (1986) Gene 43: 265-272.
16 Yother et al. (1992) J. Bacteriol., 374: 601-609
17 Romero et al. (1990) J. Bacteriol., 5064-5070.
18 Garcia et al. (1988) PNAS 85: 914-918
19 Garcia et al. (1990) Gene, 86: 81-88.
20 Diaz et al.(1992) J. Bacteriol., 174: 5516-5525.
21 Dove et al. (1990) J. Infect. Immun., 58: 480-488.
22 Wren et al. (1990) FEMS Microbiol Lett., 70: 1-6.
23 Barroso et al (1990) Nucl. Acids. Res., 18: 4004
24 von Eichel-Streiber et al. (1992) Mol. Gen. Genet., 233: 260-268.
25 Sartinger et al. (1993) EMBL Data Library.
26 von Eichel-Streiber et al. (1995) Mol Microbiol.
27 Hoffman et al. (1993) EMBL Data Library.
28 Hoffman et al. (1995) Mol. Gen.
29 Sanches et al. (1994) EMBL Data Library.
30 Sanches et al. (1995) EMBL Data Library.

In various embodiments the Kₐ for binding of the carbohydrate binding domains/proteins to cellulose is at least in the range of weak antibody-antigen extractions, i.e., at least 10³ M⁻¹, preferably at least 10⁴ M⁻¹, most preferably at least 10⁶ M⁻¹.

### Secretory Signal Sequence.

In various embodiments the peptide comprising the cell wall sorting signal (CWS) also contains a secretory signal sequence to enhance/facilitate transport through the cell membrane. Typical Gram-positive secretory signal peptides are N-terminal peptides.

Gram-positive secretion signals are well known to those of skill in the art. In certain embodiments the secretory signal sequence comprises a B. subtilis phrC secretory signal or homologues thereof.

### Gram-Positive Microorganisms.

In various embodiments it is contemplated that the display methods described herein can be used with virtually any microorganism capable of exploiting a sortase A transpeptidase reaction to anchor a protein to the cell surface. In various embodiments the microorganism is a Gram-positive microorganism (e.g., a Gram-positive bacterium).

The term “Gram-positive bacteria” generally refers to bacteria that are stained dark blue or violet by Gram staining Gram-positive microorganisms are well known to those of skill in the art. Gram-positive bacteria are generally divided into the Actinobacteria and the Firmicutes. The Actinobacteria or actinomycetes are a group of Gram-positive bacteria with high G+C ratio. They include some of the most common soil bacteria. Other Actinobacteria inhabit plants and animals and including some pathogens, such as Mycobacterium, Corynebacterium, Nocardia, Rhodococcus and a few species of Streptomyces. The majority of Firmicutes have Gram-positive cell wall structure. Illustrative Gram-positive bacteria include, but are not limited to Acetobacterium, Actinomyces (e.g., A. israelii), Arthrobacter, Bacillus (e.g., B. subtilis), Bifidobacterium, Clostridium, Clostridium spp. (e.g., C. perfringens, C. septicum, C. tetanomorphum), Corynebacterium, Enterococcus, Eubacterium, Frankia, Heliobacterium, Heliospirillum, Lactobacillus, Lactococcus, Leuconostoc, Listeria, Listeria spp., Megasphaera, Micrococcus spp., Micromonospora, Mycobacterium, Nocardia, Pectinatus, Pediococcus, Propionibacterium, Selenomonas, Sporomusa, Staphylococcus spp. (e.g., S. aureus), Streptococcus spp., (e.g., S. pneumoniae, B group streptococci), Streptomyces, and Zymophilus. Similarly, sortases, secretion signals cell wall sorting signals can include, but are not limited to, those derived from any Gram-positive microorganism.

In certain embodiments the bacterial host is selected from the group of non-pathogenic and/or non-invasive, Gram-positive bacteria consisting of Lactobacillus, Lactococcus, Pediococcus, Carnobacterium, Bifidobacterium, Oenococcus, Bacillus subtilis, Streptococcus thermophilus, and other non-pathogenic and/or non-invasive Gram-positive bacteria known in the art. In certain embodiments the bacterial host cell preferably is a Gram-positive bacterium, more preferably a Gram-positive bacterium that belongs to a genus selected from the group consisting of Lactobacillus, Lactococcus, Leuconostoc, Carnobacterium, Bifidobacterium, Bacillus, Streptococcus, Propionibacterium, Oenococcus, Pediococcus, Enterococcus. In certain embodiments the bacterial host cell is a bacterium that belongs to a species selected from the group consisting of L. acidophilus, L. amylovorus, L. bavaricus, L. brevis, L, caseii, L. crispatus, L. curvatus, L. delbrueckii, L. delbrueckii subsp. bulgaricus, L. fermentum, L. gallinarum, L. gasseri, L. helveticus, L. jensenii, L. johnsonii, L. minutis, L. murinus L. paracasei, L. plantarum, L. pontis, L. reuteri, L. sacei, L. salivarius, L. sanfrancisco, Lactobacillus ssp., C. piscicola, B. subtilis, Leuconostoc mesenteroides, Leuconoctoc lactis, Leuconostoc ssp, L. lactis subsp. lactis, L. lactis subsp. cremoris, Streptococcus thermophilus, B. bifidum, B. longum, B. infantis, B. breve, B. adolescente, B. animalis, B. gallinarum, B. magnum, and B. thermophilus.

### Methods of Engineering Microorganisms.

As indicated above, in certain embodiments, microorganisms are engineered to contain a nucleic acid construct that exploits a sortase pathway to covalently anchor a protein to the surface of the cell. In certain embodiments the nucleic acid construct encodes a protein comprising or more cellulolytic enzyme(s) (enzymatic domains) with a secretory signal sequence (e.g., at the N-terminus of the protein) and a cell wall sorting signal (e.g., at the carboxyl terminus of the protein). In certain embodiments the nucleic acid construct encodes a protein comprising one or more cohesin domains attached to a secretory signal sequence (e.g., at the N-terminus of the protein) and a cell wall sorting signal (e.g., at the carboxyl terminus of the protein).

A microorganism is transfected with the construct and as encoded protein is transcribed it is displayed on the surface of the microorganism, e.g., through the transpeptidase reaction mediated by a sortase. The sortase can be an endogenous sortase expressed by the microorganism. In certain embodiments the sortase can be a sortase that is encoded by the same or another nucleic acid construct transfected into the microorganism. In certain embodiments the sortase is a sortase found in the subject microorganism, and in certain embodiments, the sortase is a sortase characteristic of a different microorganism.

In certain embodiments, particularly where a minicellulosome is to be expressed, the same construct or a different nucleic acid construct can be provided that encodes one or more dockerins each attached to a different enzyme (e.g., cellulolytic enzyme) as described above.

Methods of making the nucleic acid constructs described herein are well known to those of skill in the art, and specific methods are illustrated in the examples. Cloning and bacterial transformation methods, DNA vectors and the use of regulatory sequences are well known to the skilled artisan and may for instance be found in Current Protocols in Molecular Biology, F. M. Ausubel et al, Wiley Interscience, 2004, incorporated herein by reference.

Many embodiments, of the methods and constructs described herein utilize an expression vector containing a nucleotide sequence that encodes the protein(s) of interest, a cell wall sorting signal and a secretion signal. Suitable expression vectors include, but are not limited to baculovirus vectors, bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, herpes simplex virus, and the like), P1-based artificial chromosomes, and any other vectors specific for specific hosts of interest. Such vectors include chromosomal, nonchromosomal and synthetic DNA sequences, and may comprise a full or mini transposon for the integration of a desired DNA sequence into the host chromosome. Examples of tranposons include but are not limited to TNS, TN7, and TN10, as well as the engineered transposomes from Epicentre (www.epicentre.com).

Numerous suitable expression vectors are known to those of skill in the art, and many are commercially available. The following vectors are provided by way of example; for bacterial host cells: pQE vectors (Qiagen), pBluescript plasmids, pNH vectors, lambda-ZAP vectors (Stratagene); pTrc99a, pKK223-3, pDR540, and pRIT2T (Pharmacia); for eukaryotic host cells: pXTI, pSGS (Stratagene), pSVK3, pBPV, pMSG, and pSVLSV40 (Pharmacia). However, any other plasmid or other vector, with or without various improvements for expression, may be used so long as it is compatible with the host cell.

In certain embodiments the subject vectors will contain a selectable marker gene. In certain embodiments this gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli, and the like.

The vector(s) of interest can be transfected into and propagated in the appropriate host. Methods for transfecting the host cells with the genomic DNA vector can be readily adapted from those procedures which are known in the art. For example, the vector can be introduced into the host cell by such techniques as the use of electroporation, precipitation with DEAE-Dextran or calcium phosphate, or lipofection.

Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter (see, e.g., U.S. Patent Publication No. 2004/0131637), apagC promoter (Pulkkinen and Miller (1991) J. Bacteriol., 173 (1): 86-93; Alpuche-Aranda et al. (1992) Proc. Natl. Acad. Sci. U.S.A. 89(21): 10079-83), a nirB promoter (Harborne et al. (1992) Mol. Micro. 6: 2805-2813), and the like (see, e.g., Dunstan et al. (1999) Infect. Immun. 67: 5133-5141; McKelvie et al. (2004) Vaccine 22: 3243-3255; Chatfeld et al. (1992) Biotechnol. 10: 888-892, and the like); a sigma70 promoter, e.g., a consensus sigma70 promoter (see, e.g., GenBank Accession Nos. AX798980, AX798961, and AX798183); a stationary phase promoter, e.g., a dps promoter, a spy promoter, and the like; a promoter derived from the pathogenicity island SPI-2 (see, e.g., WO96/17951); an actA promoter (see, e.g., Shetron-Rama et al. (2002) Infect. Immun. 70:1087-1096); an rpsM promoter (see, e.g., Valdivia and Falkow (1996). Mol. Microbiol. 22:367-378); a tet promoter (see, e.g., Hillen, W. and Wissmann, A. (1989) In Saenger, W. and Heinemann, U. (eds), Topics in Molecular and Structural Biology, Protein-Nucleic Acid Interaction. Macmillan, London, UK, Vol. 10, pp. 143-162); an SP6 promoter (see, e.g., Melton et al. (1984) Nucl. Acids Res. 12:7035-7056); and the like.

In certain embodiments the nucleic acid constructs of interest are operably linked to an inducible promoter or to a constitutive promoter. Inducible and constitutive promoters are well known to those of skill in the art.

Where the host cell is genetically modified to express two or more gene products (e.g., a sortase and a protein comprising a sorting signal), nucleotide sequences encoding the two or more gene products will in some embodiments each be contained on separate expression vectors and in some embodiments contained in the same vector.

Where nucleotide sequences encoding the two or more gene products are contained in a single expression vector, in some embodiments, the nucleotide sequences will be operably linked to a common control element (e.g., a promoter), e.g., the common control element controls expression of all gene product-encoding nucleotide sequences on the single expression vector. In some embodiments, the nucleotide sequences encoding different gene products are operably linked to different control element(s) (e.g., promoter(s)). In some embodiments, one of the nucleotide sequences will be operably linked to an inducible promoter, and one or more of the other nucleotide sequences will be operably linked to a constitutive promoter.

As described above, the nucleic acid constructs may be introduced into the host cell as extra-chromosomal genetic materials that can replicate themselves (e.g., plasmids), or as genetic material integrated into the host genome. Regardless of whether the heterologous genes are integrated into the host genome, or exist as extra-chromosomal genetic materials, the optimal expression of the constructs heterologous genes belonging to a new metabolic pathway can on occasion benefit from coordinated expression of such genes, tight control over gene expression, and consistent expression in all kinds of host cells.

Methods and systems are provided that fine-tune the expression of heterologous genes, which in turn allow reproducible manipulation of metabolism in model microbes, such as E. coli, Bacillus subtillis, and Aspergillus nidulans. These methods allow balanced expression of the heterologous genes (e.g., those encoding the cellulosome) by techniques such as fine-tuning mRNA stability, the use of inducible promoters of various strengths, etc. See, for example, Keasling et al., New tools for metabolic engineering of E. coli. In Metabolic Engineering, S.- Y. Lee and E. T. Papoutsakis, eds. Marcel Dekker, New York, N.Y. (1999); Keasling, Gene-expression tools for the metabolic engineering of bacteria. Trends in Biotechnology 17:452-460, 1999; Martin et al., Redesigning cells for production of complex organic molecules. ASM News 68: 336-343, 2002 (all incorporated herein by reference).

While the foregoing discussion and examples below focus on Gram-positive bacteria and Sortase A, it will be appreciated that the methods described herein are amenable for use in any microorganism in which a sortase is found or can be expressed and is functional. Thus, for example, sortase enzymes have also been identified in the gram-negative organisms Bradyrhizobium japonicum, Colwellia psychroerythraea, Microbulbifer degradans, Shewanella oneidensis, and Shewanella putrefasciens, as well as in Methanobacterium thermoautotrophicum, a thermophilic archaeon (Pallen et al. (2003) Curr. Opin. Microbiol. 6: 519-527.). The use of the methods described herein with any of these organisms is also contemplated.

The foregoing methods and constructs are intended to be illustrative and not limiting. Using the teachings provided herein, numerous proteins, enzymes, minicellulosomes and the like can be stably displayed on the surface of a microorganism.

### EXAMPLES

The following examples are offered to illustrate, but not to limit the claimed invention.

### Example 1

### Assembly of Minicellulosomes on the Surface of Bacillus subtilis

To cost-efficiently produce biofuels new methods are needed to convert lignocellulosic biomass into fermentable sugars. One promising approach is to degrade biomass using cellulosomes, which are surface displayed multi-cellulase containing complexes present in cellulolytic Clostridium and Ruminococcus species. In this study we created cellulolytic strains of B. subtilis that display one or more cellulase enzymes. Proteins containing the appropriate cell wall sorting signal are covalently anchored to the peptidoglycan by co-expressing them with the B. anthracis sortase A (SrtA) transpeptidase. This approach was used to covalently attach the CelA endoglucanase from C. thermocellum to the cell wall. In addition, a CelA-dockerin fusion protein was anchored on the surface of B. subtilis via non-covalent interactions with a cell wall attached cohesin module. We also demonstrate that it is possible to assemble multi-enzyme complexes on the cell surface. A three enzyme containing minicellulosome was displayed on the cell surface that consists of a cell wall attached scaffoldin protein which non-covalently binds to three cellulase-dockerin fusion proteins. B. subtilis has a robust genetic system and is currently used in a wide range of industrial processes. Thus, grafting larger, more elaborate minicellulosomes onto the surface of B. subtilis can yield cellulolytic bacteria with increased potency that can be used to degrade biomass.

### Materials And Methods

Strains and Plasmids.

B. subtilis JH642 (Perego et al. (1988) Mol. Microbiol. 2: 689-699) and BAL2238 served as parent strains to produce the strains listed in Table 6.

TABLE 6
Bacterial strains used in this study.

| | | Pheno- | Reference/ |
|---|---|---|---|
| Strain | Relevant Genotype | type^{a} | construction |
| JH642 | trpC2 pheA1 | | Perego et al. |
| | | | (1988) |
| | | | Mol. Microbiol. |
| | | | 2: 689-699 |
| BAL2238 | ΔwprA:hyg trpC2 pheA1 | | This study |
| Derived from JH642: | | | |
| TDA01 | amyE::(P_{xylA}-srtA cat)^{h} | SrtA^{b} | This study |
| TDA02 | thrC::(P_{spachy}-sp-his₆-celA-fib | celA^{c} | This study |
| | erm) | | |
| TDA03 | amyE::(P_{xylA}-srtA cat) | SrtA, | This study |
| | thrC::(P_{spachy}-sp-his₆-celA-fib | CelA^{b,c} | |
| | erm) | | |
| Derived from Bal2238: | | | |
| TDA05 | amyE::(P_{xylA}-srtA cat) | SrtA, | This study |
| | thrC::(P_{spachy}-sp-his₆-celA-fib | CelA^{b,c} | |
| | erm) | | |
| TDA06 | amyE::(P_{xylA}-srtA cat) | SrtA, | This study |
| | thrC::(P_{spachy}-sp-his6-coh-fib | Coh^{b,d} | |
| | erm) | | |
| TDA07 | amyE::(P_{xylA}-srtA cat) | SrtA, | This study |
| | thrC::(P_{spachy}-sp-his₆-coh-fib; | Coh, | |
| | sp-His₆-celA-doct erm) | CelA- | |
| | | Doct^{b,d,e} | |
| TDA08 | amyE::(P_{xylA}-FLAG-srtA cat) | SrtA, | This study |
| | thrC::(P_{spachy}-sp-his₆-celA-fib- | CelA- | |
| | gst erm) | GST^{b,f} | |
| TDA08 | amyE::(P_{xylA}-srtA cat) | SrtA | This study |
| | thrc::(P_{spachy}-sp-ha-scaf-fib | Scaf^{b,g} | |
| | cat) | | |

^{a}Proteins expressed by the strains.
^{b}Full length sortase A transpeptidase from B. anthracis str. Ames (GenBank Accession AE016897.1).
^{c}CelA surface protein consists of a secretory peptide derived from B. subtilis PhrC (sp, residues 1-35, GenBank Accession ZP_03590039), a hexahistidine tag (His₆), C. thermocellum ATCC 27405 (obtained from the ATCC) endoglucanase A (family 8 endoglucanase, celA, residues 32-434, GenBank Accession K03088), and the C terminal portion of S. aureus NCTC 8325 (fib, residues 756-917, GenBank Accession CP000253).
^{d}Coh surface protein is identical to CelA, except that a cohesin domain from C. thermocellum ATCC 27405 (coh, residues 182-328, GenBank accession ABN54273) has replaced the CelA polypeptide.
^{e}CelA-Doct protein is identical to CelA, except that fib has been replaced with a CBM and dockerin module from C. thermocellum ATCC 27405 Xyn10B (residues 540-790, GenBank accession ABN52146).
^{f}CelA-GST surface protein is identical to CelA, except that GST from plasmid pGEX-4t (GE Life Sciences) has been appended to the C-terminus.
^{g}Scaf surface protein is identical to CelA, except that the CelA polypeptide has been replaced by a three cohesin containing polypeptide (type I cohesins from C. thermocellum CipA, C. cellulolyticum CipC, and R. flavefaciens ScaB) and a family 3 CBM (Fierobe et al. (2005) J. Biol. Chem. 280: 16325-16334).
^{h}erm, ethromycin; cat, chloramphenicol acetyltransferase; amyE, these genes have been integrated into the amyE locus in the chromosome; thrC, these genes have been integrated into the thrC locus in the chromosome.

BAL2238 was created by transforming JH642 with the ΔwprA::hyg allele from WB800 (Wu et al. (2002) Appl. Environ. Microbiol., 68: 3261-3269). The full-length srtA gene from Bacillus anthracis str. Ames was cloned downstream from a xylose inducible promoter and integrated into the Bacillus were transformed with XhoI linearized pSrtA and plated on LB agar 140 containing 5 μg/ml chloramphenicol. Allelic replacement of amyE with PxylA-srtA and chloramphenicol acetyltransferase (cat) was confirmed by PCR amplification of chromosomal DNA and sensitivity to 10 μg/ml spectinomycin. Isopropyl β-D-1-thiogalactopyranoside (IPTG) inducible genes encoding proteins that can be anchored to the cell wall by SrtA were inserted into the thrC locus using standard methods and E. coli-B. subtilis shuttle plasmid pBL112 (Lanigan-Gerdes et al. (2007) Mol. Microbiol. 65: 1321-1333). The nucleotide sequences of the primers used to generate plasmids used in this study are shown in Table 7. Table 6 lists the specific strains that were generated, including the gene names and accession codes, as well as protein amino acid numbers. E. coli strain XL2Blue [recA1 endA1 gyrA96 thi-1 hsdR17 supE44 relA1 lac [F′ proAB lacIqZΔM15 Tn10 (Tet′) Amy Cam′] was used as the host for all genetic manipulations outside of B. subtilis. Expression plasmids producing CelG-Docf (pETGf, C. cellulolyticum endoglucanase CelG fused to the type I dockerin from R. flavefaciens) and CelE-Docc (pETEc, C. cellulolyticum exoglucanase CelE fused to its native dockerin) have been described previously (Fierobe et al. (2005) J. Biol. Chem. 280: 16325-16334). A pET28a-based plasmid producing CelA-Doct (residues 32-434 of CelA from C. thermocellum fused at its C-terminus to residues 540-790 of the dockerin from the Xyn10B protein) was created for this study using standard subcloning methods and primers listed in Table 7. For cloning, E. coli and B. subtilis cultures were grown in Luria-Bertani (LB) medium supplemented with the appropriate antibiotic (100 μg/ml ampicillin, 1 μg/ml erythromycin, 5 μg/ml chloramphenicol, 50 μg/mL kanamycin or 100 μg/ml hygromycin B).

TABLE 7
Oligonucleotides used.

| | | SEQ |
|---|---|---|
| | | ID |
| Name | Description/Sequence | NO |
| SrtAF | 5′ end of srtA of B. anthracis | 21 |
| | gacagAAGCTTaaaggagtgaaggtttgtatgaataagcaaagaatttatag | |
| SrfAR | 3′ end of srtA of B. anthracis | 22 |
| | gacagGGATCC ttatttatcatcatcatctttataat ctttcttcgc | |
| PhrCF | 5′ end of phrC of B. subtilis | 23 |
| | gacagAAGCTT aaaggagtgaaggtttgtatg | |
| PhrCR | 3′ end of phrC of B. subtilis | 24 |
| | gacagACTAGTtgtcacatgaaagtcgagtg | |
| HisCelAF | 5′ end of celA of C. thermocellum | 25 |
| | gacagACTAGTcatcatcatcatcatcatcat gcaggtgtgccttttaacacaaaatac | |
| HisCelAR | 3′ end of celA of C. thermocellum | 26 |
| | gacagGCGGCCGCataaggtaggtggggtatgc | |
| FibF | 5′ end of the c-terminal domain of fibronectin binding | 27 |
| | protein B of S. aureus | |
| | gacagGCGGCCGCccaagtaagtggtcataatgaaggtc | |
| FibR | 3′ end of the c-terminal domain of fibronectin binding | 28 |
| | protein B of S. aureus | |
| | gacagGCATGCttatgctttgtgattctttttatttctgcg | |
| CohF | 5′ end of the second cohesin of cipA of C. thermocellum | 29 |
| | gacagACTAGT catcatcatcatcatcatggtgtggtagtagaaattgg | |
| CohR | 3′ end of the second cohesin of cipA of C. thermocellum | 30 |
| | gacagGCGGCCGCcttggtcggtgttgcattgc | |
| DoctF | 5′ end of the CBM and dockerin module of xyn10B of C. | 31 |
| | thermocellum | |
| | gacagGCGGCCGCCcagtccgaatggggcgacggtaa | |
| DoctR | 3′ end of the CBM and dockerin module of xyn10B of C. | 32 |
| | thermocellum | |
| | gacagGCATGCttaaggattttctgctacagg | |
| CelA- | 5′ end of the celA-doct fusion protein | 33 |
| DoctF | gacagGCGGCCGCCcagtccgaatggggcgacggtaa | |
| CelA- | 3′ end of the cela-doct fusion protein | 34 |
| DoctR | gacaGCTAGCgcaggtgtgccttttaacac | |
| GSTF | 5′ end of gst of pGES-4t containing 20 nt from fibronectin | 35 |
| | binding protein B of S. aureus | |
| | Gaaataaaaagaatcacaaagcaatgtcccctatactaggttattgg | |
| GSTR | 3′ end of gst of pGEX-4t containing 20 nt from fibronectin | 36 |
| | binding protein B of S. aureus | |
| | gacagGCATGC ttagtcacgatgcggccgctcg | |
| FibGSTR | 3′ end of fibronectin binding protein B of S. aureus | 37 |
| | containing 20 nt of gst of pGEX-4t | |
| | ccaataacctagtataggggacattgctttgtgattctttttatttc | |
| ScafF | 5′ end of fusion scaffold containing a type I cohesin | 38 |
| | of C. cellulolyticum cipC, a type I cohesin and CBM | |
| | of C. thermocellum cipA, and a type I cohesin of scaB | |
| | of R. flavefaciens | |
| | gacagACTAGT tacccatacgatgttccagattacgct | |
| | ggcgattctcttaaagttacag | |
| ScafR | 3′ end of fusion scaffold containing a type I cohesin | 39 |
| | of C. cellulolyticum cipC, a type I cohesin and CBF | |
| | of C. thermocellum cipA, and a type I cohesin of scaB | |
| | of R. flavefaciens | |
| | gaacagGCGGCCGC cttaacaatgatagcgcc | |

Restriction sites are in uppercase. Ribosomal binding sites used are in bold. Nucleotide sequence for FLAG tag is italicized and underlined. Nucleotide sequence for polyhistidine tag is underlined. Nucleotide sequence for HA tag is italicized.

### Immunofluorescence Microscopy.

Strains TDA02 (CelA expressing) and TDA03 (CelA and SrtA expressing) were used. A 5 ml culture of each strain was grown overnight in LB media supplemented with the 1 μg/ml erythromycin. One-half milliliter of the culture was then used to inoculate 50 ml of fresh LB medium (1/100 dilution). The 50 ml cultures were then shaken at 37° C. until they reached an OD₆₀₀ of 0.2. CelA expression was then induced by adding IPTG to a final concentration of 1 mM. The culture containing strain TDA03 was also induced to express SrtA by adding xylose to the culture when its OD600 reached 0.1 (final xylose concentration of 0.5%). When all cell cultures reached an OD600 of 2.0, they were centrifuged at 3000×g for 5 min and then re-suspended in 1 ml of Phosphate Buffered Saline (PBS, 8 g/liter NaCl, 0.2 g/liter KCl, 1.44 g/liter Na₂HPO₄, 0.24 g/liter KH₂PO₄, pH 7.4). The cells were then centrifuged and the pellet re-suspended in 800 μl of PBS and 200 μl of Fix buffer (12% formaldehyde, 150 mM NaH₂PO₄). This solution was incubated at room temperature for 15 min, and then placed on ice for 1 hr. After centrifugation at 3000×g for 5 min, the pellet was re-suspended in 1 ml of PBS. This washing step was repeated for a total of 3 times. The final pellet obtained from this process was then re-suspended in a volume of GTE buffer (25 mM Tris-HCl, pH 8.0, 10 mM EDTA, 50 mM glucose) such that the OD₆₀₀ was ˜1.0. Twenty microliters of suspended cells was then aspirated onto a poly-lysine coated microscope slide and dried. The slides were then blocked by adding a 2% solution of Bovine Serum Albumin (BSA) protein dissolved in PBS buffer. After incubating for 15 min, the slides were washed with PBS. CelA display was probed using an anti-His6 immunoglobulin G antibody (1.25 μg/μl, Abgent, San Diego, Calif.). After incubating for 1 hr, the slides were washed with PBS and incubated for 1 hr with goat anti-mouse immunoglobulin G conjugated with Dylight 488 (0.2 ng/μl, Fisher Scientific). After washing the slides again with PBS, a 10 μl solution containing 70% glycerol and 5 μg/ml 4′,6-diamidino-2-phenylindole (DAPI) was added prior to imaging. Data was collected on an Applied Precision Delta Vision Deconvolution microscope (457 nm and 528 nm excitation was used for Dylight 488 and DAPI, respectively).

Whole Cell Cellulase Assays.

For each B. subtilis strain a 5 ml LB culture containing 1 μg/ml erythromycin was grown overnight. A total of 0.5 ml from each overnight culture was then added to a 50 ml LB solution containing 1 μg/ml erythromycin. Cells were then shaken at 37° C. until they reached an OD₆₀₀ of 0.1. If SrtA expression was desired then xylose was added to a final concentration of 0.5%. All cultures were then grown to an OD₆₀₀ of 0.2, at which point IPTG was added to a final concentration of 1 mM to induce the expression of the surface displayed protein. Three milliliter samples of each culture were taken periodically, to measure their OD₆₀₀ and enzymatic activity. To measure enzymatic activity, the 3 ml sample was centrifuged for 5 min at 3000×g and the pellet was re-suspended in assay buffer (20 mM Tris-HCl, pH 6.0). The cells were then centrifuged again, and the pellet was resuspended in 2 ml carboxymethyl cellulose (0.5% CMC, medium viscosity (Sigma), 20 mM Tris-HCl, pH 6.0). Each cell suspension was then incubated at 37° C. for 30 min, and centrifuged at 20,000×g for 1 min. Activity was determined by adding 3 ml of dinitrosalicylic acid (DNSA) to the supernatant (the DNSA solution contained: 1% DNSA, 1% NaOH, 0.2% phenol, and 0.05% Na₂SO₃). Samples were then boiled for 10 min and the absorbance was recorded at 575 nm. The amount of sugar released was quantified using a glucose standard curve. All whole cell enzymatic assays were performed in triplicate. To control for different growth rates, the enzymatic activity values obtained for each 3 ml culture was normalized by dividing this data by the OD₆₀₀ value determined for each culture prior to centrifugation.

Immunoblot Analysis of Cell Fractions.

Samples used to monitor protein expression were created in an identical manner as samples used to monitor whole cell cellulase activity (described above). In this work, the 50 ml cultures were grown for 3 hrs after the addition of IPTG and then centrifuged for 5 min at 3,000×g. The cell pellet was then re-suspended in 1 mL STM buffer (25% sucrose, 50 mM Tris-HCl pH 8.0, 5 mM MgCl₂) and re-centrifuged. The cell pellets were then re-suspended in a volume of STM, such that each had an OD₆₀₀ value of 1 (typically 1 ml of STM was used). The STM solution also contained lysozyme enzyme at a final concentration of 500 μg/ml. The re-suspension was incubated at 37° C. for 30 min, and then centrifuged for 10 min at 20,000×g. The supernatant contains solubilized cell wall proteins and was subjected to immunoblot analysis. The pellet contains protoplasts, whose proteins were released by re-suspending the pellet in 0.1N NaOH such that the solution had an OD₆₀₀ of ˜1. The protoplast solution was then centrifuged for 10 min at 20,000×g. The membrane and cytoplasmic proteins were collected in the supernatant after centrifugation. To precipitate proteins that had been secreted into the growth medium, trichloroacetic acid (TCA) was added to the LB supernatant obtained by centrifuging the 50 ml cell culture (final concentration of 10% w/v TCA). The solution was then centrifuged and the pellet was re-dissolved in water for immunoblot analysis. The solutions containing the cell wall, protoplast and secreted protein fractions were then separated by sodium dodecyl sulfate polyacrylamide gel electrophoresis (SDS-PAGE) and transferred to a polyvinylidene fluoride (PVDF) membrane using standard procedures. The membrane was then blocked by soaking it for 1.5 hrs in Tris Buffered Saline Plus Tween (TBST, 20 mM Tris-HCl, pH 7.5, 500 mM NaCl, 0.05% Tween 20) supplemented with 5% BSA. The membrane was then incubated with anti-His6 immunoglobulin G (0.25 μg/μl) for 1 hr, washed with TBST for 30 min and incubated with a horseradish peroxidase (HRP) conjugated rabbit anti-mouse immunoglobulin G secondary antibody (1:50,000 dilution for 1 hr., Sigma, cat. No. A9044). The blot was then washed and incubated with Pierce ECL Western Blotting substrate (0.125 ml/cm²) for 1 min and visualized by exposing to an autoradiography film (Fisher Scientific). A similar immunoblot analysis was performed using strain TDA08 that expressed the CelA-GST protein (Table 6, FIG. 4, panel C) to track the fate of the processed and unprocessed protein using an anti-His₆ immunoglobulin G primary antibody. The details used to perform this procedure have been described previously (Budzik et al. (2008) J. Biol. Chem. 283: 36676-36686).

Protein Purification and Complex Assembly on the Cell Surface.

The CelA, CelA-Doct, CelE-Docc, and CelG-Docf proteins were expressed in E. coli and purified to homogeneity. Methods used to produce CelE-Docc and CelG-Docf have been described previously (Fierobe et al. (2005) J. Biol. Chem. 280: 16325-16334). The histidine tagged CelA and CelA-Doct were produced from a 1 L culture of LB and purified as specified by Novagen and using a Co-NTA resin (HisPur cobalt resin, Fisher Scientific). Purified enzymes were dissolved in binding buffer (25 mM Tris-HCl, pH 7.0, 200 mM NaCl, 5 mM CaCl₂). For in vitro cellulase assays, purified CelA (32-434) was dissolved in assay buffer (20 mM Tris-HCl, pH 6.0). Minicellulosomes were constructed on the surface of B. subtilis by incubating purified enzymes with cells containing either Coh or Scaf attached to their cell wall. Procedures used to display these proteins are identical to those described above. A 50 ml culture of cells displaying either Coh or Scaf were grown for varying amounts of time in the presence of 1 mM IPTG. A 3 ml sample of each culture was then centrifuged and resuspended in binding buffer. The solution was then centrifuged for a second time and resuspended in 25 μl of binding buffer containing 100 μM purified cellulase enzyme (CelA-Doct, CelE-Docc, and/or CelG-Docf). After incubating on ice for 1 hr, the suspensions were centrifuged to remove unbound protein. The cells were then re-suspended in binding buffer and centrifuged at 3,000×g for 5 min. This washing step was repeated 3 times. The pellets were then re-suspended in 2 ml of 0.5% CMC or 0.5% HCl-treated amorphous cellulose. HCl-treated amorphous cellulose was prepared as described by Hsu and Penner, except that Whatman No. 1 filter paper was substituted for Avicel PH101 (Hsu and Penner (1991) J. Agricultural and Food Chem., 39: 1444). The amount of sugar released was determined as described above.

### Results

Construction of a Sortase Mediated Protein Display System in B. subtilis.

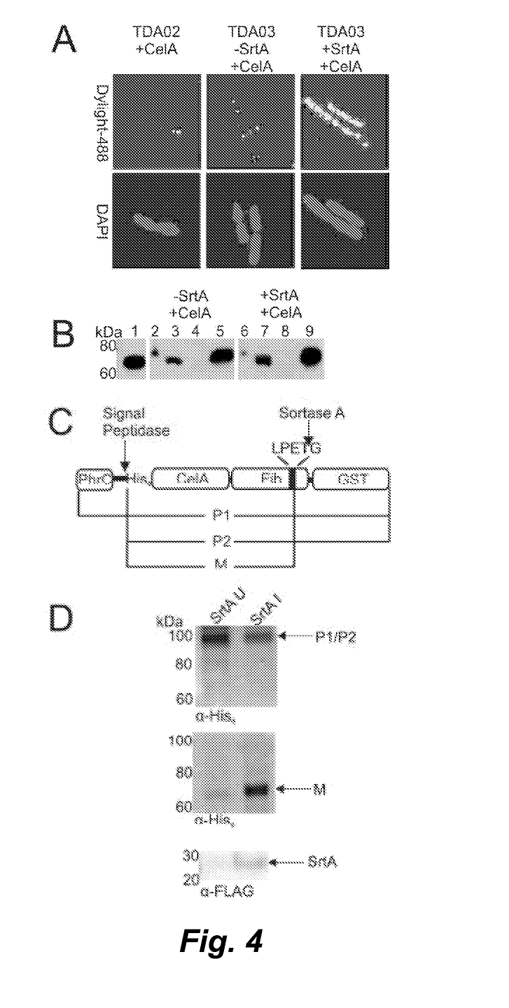

We explored whether the sortase enzyme mechanism could be used to attach an artificial minicellulosome to the surface of B. subtilis. Towards this goal we developed a system in which to anchor proteins to the peptidoglycan. The display system works by co-expressing the B. anthracis sortase A enzyme (SrtA) with a protein that it covalently anchors to the cell wall (FIG. 3, panel A) (Gaspar et al. (2005) J. Bacteriol. 187: 4646-4655). This enzyme was chosen because its second substrate, lipid II, is conserved between B. subtilis and B. anthracis, suggesting that SrtA could function properly in both organisms (Marraffini et al. (2006) Microbiol. Mol. Biol. Rev. 70: 192-221). Homologous recombination was used to introduce the srtA gene into the amyE locus under the control of a xylose inducible PxylA promoter. The protein to be anchored by SrtA was introduced through similar methods into the thrC locus and is expressed from an IPTG inducible Pspachy promoter. Appended to the beginning of the protein substrate is the N-terminal secretory peptide derived from the B. subtilis PhrC protein and a hexahistidine (His₆) tag. The protein also contains at its C-terminus a portion of the Staphylococcus aureus Fibronectin Binding Protein B, which consists of a 123 amino acid spacer segment and a cell wall sorting signal (CWS) (FIG. 3, panel B). This S. aureus CWS sequence is identical to those found in many B. anthracis surface proteins that are anchored to the cell wall by SrtA (Gaspar et al. (2005) J. Bacteriol. 187: 4646-4655).

Initially, the CelA endoglucanase from C. thermocellum (FIG. 3, panel B) was displayed on the surface of B. subtilis. CelA was used because its in vitro activity has been well characterized and because it has previously been displayed on the surface of yeast (Tsai et al. (2009) Appl. Environ. Microbiol. 75: 6087-6093). Homologous recombination was used to construct strain TDA03, which expresses srtA and celA under the inducible control of xylose and IPTG, respectively. Following protein induction, the cells were grown to an OD₆₀₀ of 2.0 and protein display was visualized using immunofluorescence microscopy (FIG. 4, panel A). Antibody staining of the N-terminal hexahistidine tag within the CelA protein reveals that it is located on the bacterial surface (right panel, FIG. 4, panel A). Cells in control experiments in which SrtA expression was not induced showed significantly smaller amounts of displayed enzyme (center panel, FIG. 4, panel A). Moreover, minimal display is observed for strain TDA02, which lacks the srtA gene (FIG. 4, panel A, left panel). This indicates that sortase is required to display CelA and that the expressed CelA protein does not associate with the cell surface non-specifically in the absence of sortase.

To substantiate that CelA is linked to the cell wall we fractionated TDA03 cells and performed immunoblot experiments to determine its location. As shown in FIG. 4, panel B, a significant amount of CelA is localized to the cell wall when both SrtA and CelA are expressed. In particular, when whole cells are washed and treated with lysozyme, significant amounts of protein are released from the cell wall (lane 7). However, no detectable CelA is found in the cytoplasm or membrane fractions (lane 8). Furthermore, only a small amount of CelA is observed when whole cells are lysed, presumably because most of the protein is attached to the cell wall and thus cannot be separated by SDS-PAGE (lane 6). Interestingly, the display system is not 100% efficient, as a significant amount of CelA is secreted into the supernatant (lane 9). Additional control experiments were performed in which SrtA expression was not induced. Unexpectedly, this work yielded generally similar results and showed that CelA is targeted to the cell wall in the absence of SrtA, albeit at reduced levels (lane 3). This suggests that some of the CelA protein produced in the absence of SrtA non-specifically binds to the cell wall. However, this protein is not functional (see below) and it is not exposed to the growth medium based on our inability to detect it by immunofluorescence microscopy (FIG. 4, panel A).

To more thoroughly investigate the sortase anchoring reaction, we performed additional experiments that measured the abundance of a CelA fusion protein in which glutathione-s transferase (GST) is positioned downstream from the CWS (FIG. 4, panel C) (Budzik et al. (2008) J. Biol. Chem. 283: 36676-36686). Based on its molecular weight, the addition of GST allows the mature form of the protein to be distinguished from precursors of the protein in which either the signal peptide or CWS have been cleaved. These precursors correspond to forms of the protein that have not been processed by SrtA and include the intact protein (P1) and protein in which only the N-terminal signal peptide has been cleaved by the signal peptidase (P2). As shown in the top panel of FIG. 4, panel D, when SrtA is not expressed, unprocessed CelA accumulates in the membrane and cytoplasmic fractions of B. subtilis and little mature CelA is located in the cell wall (middle panel). In contrast, when SrtA is expressed, precursor forms of CelA are diminished and mature CelA is found in the cell wall. Importantly, mature CelA appears to be covalently anchored to the cell wall, since the cell wall fraction was washed extensively with SDS prior to treatment with mutanolysin to cleave the glycan. It should be noted that data presented in FIG. 4, panel B and 4, panel D are not contradictory, since the cell wall preparations analyzed in FIG. 4, panel D were subjected to SDS treatment to stringently remove non-covalently bound protein, whereas the cell wall fractions analyzed in FIG. 4, panel B were not.

Functional Endoglucanase is Displayed on the Surface of B. subtilis.

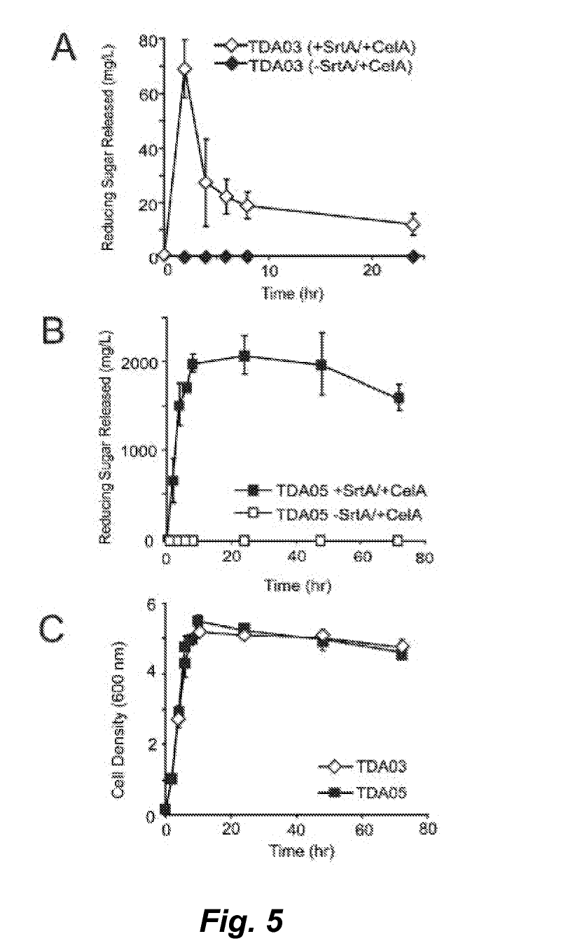

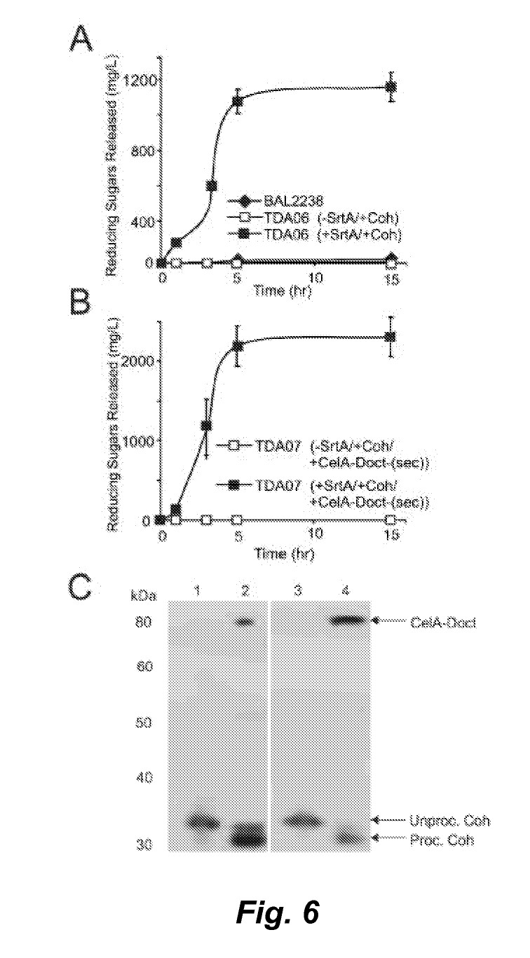

Strain TDA03 displaying CelA was tested for its ability to degrade carboxymethyl cellulose (CMC). Samples from cultures expressing SrtA and CelA were collected periodically, washed, and their cellulolytic activity determined using a standard dinitrosalicylic acid assay (Tsai et al. (2009) Appl. Environ. Microbiol. 75: 6087-6093). When both SrtA and CelA are induced, the cells degrade CMC, releasing up to 70 mg/L of reducing sugar (FIG. 5, panel A, open diamonds). The cellulolytic activity is due to cell wall attached CelA as cultures in which SrtA is not induced with xylose do not efficiently degrade CMC (FIG. 5, panel A, closed diamonds). This indicates that the cellulolytic activity of strain TDA03 is dependent on the presence of SrtA. Moreover, it suggests that residual CelA protein retained in the cell wall when SrtA is not induced (FIG. 4, panel B, lane 3) is not competent to degrade CMC, presumably because it is not sufficiently exposed or improperly folded. Finally, the cellulolytic activity of TDA03 is not caused by an endogenous B. subtilis cellulase, as strain TDA01 lacking celA does not degrade CMC (data not shown).

Inspection of FIG. 5, panel A, reveals that maximal cellulolytic activity is achieved ˜3 hours after induction of CelA expression. The activity then decreases substantially, with only 20% remaining after 6 hours. A growth analysis reveals that cells exponentially growing have maximal enzymatic activity and that the activity decreases as they transition into the stationary phase (FIG. 5, panel C). As B. subtilis expresses several extracellular proteases, we wondered whether CelA was being proteolyzed (Wen et al. (2010) Appl. Environ. Microbiol. 76: 1251-1260). To investigate this issue, whole cell activity experiments were repeated, but tetracycline was added to the cultures three hours post-induction to stop the production of proteases. This treatment preserved cell wall associated CelA activity (data not shown). The B. subtilis cell wall associated WprA protease could be degrading CelA because it is expressed throughout growth and it is well known that it degrades heterologously expressed proteins (Lanigan-Gerdes et al. (2007) Mol. Microbiol. 65: 1321-1333; Margeot et al. (2009) Curr. Opin. Biotechnol. 20(3): 372-380; Stein (2005) Mol. Microbiol., 56: 845-857; Wilson (2009) Curr. Opin. Biotechnol. 20: 295-299). We therefore introduced the cell wall attachment system into a wprA-background (B. subtilis str. BAL2238) to create strain TDA05. These cells show increased enzyme activity and stability relative to TDA03 cells (FIG. 5, panel B, closed boxes). In particular, the maximal activity obtained is 30-fold greater than that of TDA03 and only modestly decreases after 70 hours, even throughout stationary growth (FIG. 5, panel C). We were unable to determine if cells displaying CelA can grow on HCl-treated amorphous cellulose because the current system requires the addition of xylose to induce protein expression and xylose can be used by B. subtilis as a carbon source. Combined, these data indicate that CelA is attached to the cell wall by SrtA and that the deletion of the WprA protease dramatically increases cell wall attached enzyme activity.

Assembly of a Functional Surface Displayed Cohesin-Cellulase Complex Via Transcomplementation or Co-Expression.

We next determined the feasibility of assembling a cohesin-cellulase complex on the surface of B. subtilis. A protein containing a cohesin module (Coh) was anchored to the cell wall, and its ability to tether a fusion protein containing the CelA enzyme and a type I dockerin (CelA-Doct) was investigated. The Coh protein corresponds to the second cohesin module from the C. thermocellum scaffoldin protein CipA and contains the appropriate N- and C-terminal sequence elements for SrtA mediated anchoring to the cell wall (FIG. 3, panel B). CelA-Doct contains the aforementioned CelA enzyme with the type I dockerin from the C. thermocellum Xyn10B xylanase fused to its C-terminus. The Xyn10B derived polypeptide also contains a family-22 carbohydrate-binding module (CBM) (FIG. 3, panel C). This fragment of the Xyn10B polypeptide was chosen because it has previously been shown to bind with high affinity in vitro to the CipA cohesin module (Pinheiro et al. (2009) Biochem. J. 424: 375-384). The CBM of Xyn10B may not be optimal for cellulose binding as it is a family-22 CBM whose members typically bind to xylan. It should also be noted that native CelA encodes a dockerin module that can be bound by the cohesin of CipA, but it was not used because its binding specificity has not been as well characterized as the dockerin from Xyn10B (Id.). The wprA-strain TDA06 expressing SrtA and Coh was grown for varying lengths of time and the cells were then harvested by centrifugation. The cells were then re-suspended in a binding buffer containing 100 μM purified CelA-Doct protein. After washing, the ability of the cells to degrade CMC (after 30 minutes of incubation) was determined.

As shown in FIG. 6, panel A, supplying purified CelA-Doct to cells that are displaying Coh on the surface yields functional cellulolytic complexes. The cellulolytic activity is correlated with the stationary phase of growth, as it is maximal when CelA-Doct is supplied to TDA06 cells that have been expressing Coh and SrtA for more than ten hours. A maximum of 1,200 mg/L reducing sugar is released after five hours of Coh and SrtA expression, and remains stable for at least an additional ten hours. Importantly, cultures in which SrtA expression is not induced with xylose show minimal activity after incubation with purified CelA-Doct (TDA06 (−SrtA/+Coh)). A similar result is also obtained for the isogenic control strain BAL2238 that does not contain the srtA and coh genes. Immunoblot analysis was used to further substantiate that the Coh:CelA-Doct complex had assembled on the cell surface. TDA06 cultures were harvested and exposed to CelA-Doct as previously described. After washing, the cells were treated with lysozyme to release the cell wall attached Coh:CelA-Doct complex. Since both CelA-Doct and Coh contain an N-terminal His₆ tag their presence in the complex was detected using an anti-His₆ antibody. As shown in FIG. 6, panel C, when SrtA and Coh are expressed, the cell walls contain both CelA-Doct and Coh (lane 2). However, when SrtA is not expressed by the cells, only Coh is detected in the cell wall (lane 1). This species migrates at a higher molecular weight than the SrtA dependent band, and presumably corresponds to non-specifically bound protein in which the C-terminal CWS has not been cleaved. The lack of enzymatic activity in cells not expressing SrtA suggests that this non-specifically bound form of Coh is incapable of productively interacting with CelA-Doct. Thus, functional CelA-Doct can only be tethered to the cell surface via non-covalent interactions with Coh that is covalently attached to the cell wall by SrtA.

To avoid having to add purified enzymes to Coh displaying cells, we investigated whether the Coh:CelA-Doct complex could be assembled on the cell surface by co-expressing its components. Strain TDA07 was generated in which the Coh and CelA-Doct-(sec) proteins are co-expressed as a single transcript under the control of IPTG. CelA-Doct-(sec) and CelA-Doct are identical, except CelA-Doct-(sec) contains an N-terminal signal sequence that enables it to be secreted from the cell. Cultures of TDA07 expressing SrtA, Coh and CelA-Doct-(sec) possess ˜2-fold more cellulolytic activity than cells in which the Coh:CelA-Doct complex was produced by adding purified CelA-Doct (compare FIG. 6, panels A and B, closed boxes). As much as 2,300 mg/L of reducing sugar is released using strain TDA07 within five hours of protein induction and the activity remains stable for at least fifteen hours. Assembly of the Coh:CelA Doct-(sec) complex depends on sortase, as cells are unable to degrade CMC when only the Coh and CelA-Doc-(sec) proteins are expressed (FIG. 6, panel B, open boxes). An immunoblot of the cell wall fraction of TDA07 further substantiates that the complex is assembled in a sortase418 dependent manner (FIG. 6, panel C, lanes 3 and 4). Interestingly, compared to complexes created by transcomplementation, co-expressing the components increases the amount of cell wall associated CelA-Doct-(sec), which may explain why these cells exhibit greater cellulolytic activity.

Display of a Functional Minicellulosome.

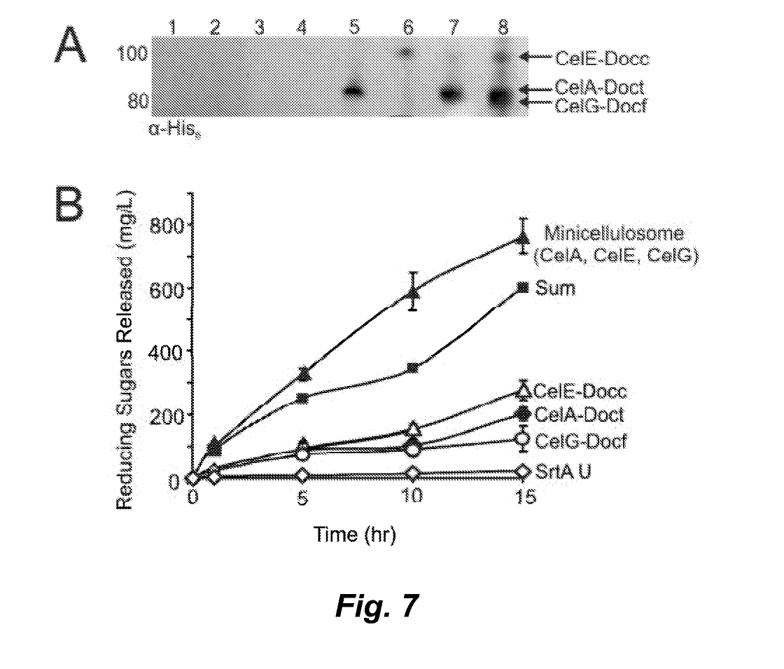

We next investigated whether it was possible to display a functional minicellulosome on the surface of B. subtilis that contained three different cellulase enzymes. The minicellulosome possesses a scaffoldin (Scaf) that contains three cohesin modules that have distinct binding specificities: (1) the 426 cohesin from the C. thermocellum CipA protein (Coht), (2) the cohesin from C. cellulolyticum CipC1 (Cohc), and (3) the cohesin from R. flavefaciens ScaB (Cohf) (FIG. 3, panel B). It also contains the family-3 CBM from C. thermocellum CipA which binds cellulose, as well as a C-terminal CWS that enables it to be anchored to the cell wall by SrtA. Scaf was used because it had previously been shown to successfully assemble a minicellulosome both in vitro and on the surface of yeast (Fierobe et al. (2005) J. Biol. Chem. 280: 16325-16334; Tokatlidis et al. (1991) FEBS Lett. 291: 185-188). Cells were induced to co-express Scaf and SrtA, and then grown for varying amounts of time. They were then centrifuged, and the cell pellets were re-suspended in solutions that contained different purified cellulase proteins. The C-termini of each cellulase protein is fused to a distinct dockerin module. Three purified cellulase-dockerin fusions were added which should each bind to a distinct cohesin module within Scaf (FIG. 3, panel C). These include: (1) the aforementioned CelA-Doct protein that binds to the Coht module, (2) CelE-Docc, that contains the C. cellulolyticum exoglucanase CelE enzyme and its native dockerin that binds to the Cohc module, and (3) CelG-Docf, that contains the CelG endoglucanase from C. cellulolyticum fused to a dockerin module from the R. flavefaciens ScaA protein which binds to the Cohf module. In separate experiments, cells displaying Scaf were incubated with each of the cellulase-dockerin proteins and subjected to immunoblot analysis that confirmed enzyme binding to Scaf (FIG. 7, panel A, lanes 5-7). In addition, an immunoblot of cells incubated with all three fusion proteins is compatible with the enzymes interacting with Scaf on the cell surface to form a minicellulosome (lane 8). As expected, association of each fusion protein with the cell wall is dependent upon the presence of SrtA anchored Scaf (lanes 1-4).

Cells displaying a minicellulosome, as well as single enzymes, were tested for their ability to degrade HCl-treated amorphous cellulose (FIG. 7, panel B). The methods used to determine cell associated enzymatic activity were identical to those used to study the surface associated CelA-cohesin complex (FIG. 6). Separate incubation of Scaf displaying cells with each cellulase-dockerin fusion protein yields similar overall activity (˜100-200 mg/L sugar produced). However, when all three enzymes are incubated with Scaf displaying cells, a ˜4 fold increase in activity is observed (˜800 mg/L sugar produced). Interestingly, the enzymes appear to be working synergistically, as the activity of cells containing a minicellulosome with all three enzymes is greater than the sum of the enzymatic activities of cells harboring only a single enzyme (˜1.3 fold more active). Importantly, the activity differences are due to the amount of displayed enzyme on each cell as the cell density of each sample tested is identical. Taken together, these data indicate that a minicellulosome containing three enzymes can assemble on the surface of B. subtilis and that these cells are more cellulolytic than cells that display only a single enzyme. Future experiments will characterize in greater detail whether the enzymes in this complex function synergistically.

### DISCUSSION

Cellulosic biomass is the most abundant source of carbon in the biosphere and it could function as an inexpensive feedstock to produce biofuels if improved methods were developed to degrade it into metabolically accessible sugars (Carroll and Somerville (2009) Annu. Rev. Plant. Biol. 60: 165-182; Chang (2007) Curr. Opin. Chem. Biol. 11: 677-684; Gomez et al. (2008) New Phytol., 178: 473-485; Margeot et al. (2009) Curr. Opin. Biotechnol. 20(3): 372-380; Rubin (2008) Nature 454: 841-845).

We explored whether B. subtilis, an industrially useful microbe that has an established genetic system, could be engineered to degrade biomass by using a sortase enzyme to display minicellulosomes on its surface. Towards this goal, we initially constructed B. subtilis cells that display the CelA cellulase from C. thermocellum. The CelA enzyme is covalently anchored to the peptidoglycan by co-expressing it with the B. anthracis Sortase A transpeptidase (SrtA). SrtA mediates the display of CelA on the surface of B. subtilis as evidenced by immunofluorescence microscopy, immunoblot analyses and the ability of the cells to degrade CMC. Both the stability and enzymatic activity of surface displayed CelA is improved when the cell wall WprA protease is genetically deleted. The improvement is substantial, with nearly 50% more protein anchored to each cell as compared to cells containing a full-complement of proteases (Nguyen and Schumann (2006) J. Biotechnol. 122: 473-482). We estimate that ˜300,000 CelA proteins may be displayed per cell. This estimate was made by measuring the enzymatic activity of cultures in which the colony forming units had been experimentally determined. It also assumes that the cell wall attached proteins will have similar enzymatic activity as the purified enzyme whose specific activity was determined experimentally (data not shown). Without being bound to a particular theory, it is believed that strains in which additional proteases are deleted will exhibit better protein display properties.

Although SrtA anchors large amounts of protein to the cell wall, the process appears inefficient as ˜70% of the expressed CelA protein is secreted into the medium. In marked contrast, B. anthracis SrtA is highly efficient in its native organism, anchoring nearly all of its protein substrates to the cell wall and very little protein is secreted (Gaspar et al. (2005) J. Bacteriol. 187: 4646-4655). The inefficiency of the B. anthracis enzyme in B. subtilis could be caused by the over expression of the protein substrate relative to the sortase enzyme. This is supported by an immunoblot analysis which revealed that even in the presence of SrtA, unprocessed CelA precursors are present. It is also possible that the protein substrates are missing features not yet identified that are required for enzyme activity. The genome of B. subtilis contains two putative sortase encoding genes whose functions have not been characterized (Nguyen and Schumann (2006) J. Biotechnol. 122: 473-482). As functional CelA protein is not displayed when SrtA is absent, these endogenous enzymes are presumably unable to anchor CelA to the cell wall. The reason for this is not known, but it could be because the endogenous sortases are not expressed during the growth conditions used in our experiments and/or the enzymes are unable to recognize the cell wall sorting signal present in the CelA substrate.

Cellulose derived from biomass is significantly more complex and heterogeneous than HCl-treated amorphous cellulose. In order to efficiently degrade it using B. subtilis, multiple enzymes can be displayed on its surface (e.g. endoglucanases, exoglucanases, β-glucosidases, xylanases, and pectinases). Towards this objective we investigated whether it was possible to assemble a cohesin:cellulase complex on the surface of B. subtilis. The cohesin:cellulase complex (Coh:CelA-Doct) is formed by covalently attaching a cohesin module to the cell wall, which in turn coordinates the non-covalent binding of a CelA-dockerin fusion protein (CelA-Doct). The complex can be assembled by either co-expressing the components or by adding purified CelA-Doct to cells displaying Coh.

Interestingly, co-expression yields cells that have ˜2-fold more enzymatic activity. The reason for this is unknown, but could occur if the CelA-Doct proteins produced in E. coli are less active, or if complex assembly is initiated as the proteins are secreted. Importantly, we also demonstrated that a similar strategy can be used to assemble a three enzyme minicellulosome. In this system, cells anchoring a scaffoldin attached to their peptidoglycan coordinate the binding of three distinct enzymes via dockerin modules. Bacterial cells displaying minicellulosomes exhibit increased activity against HCl-treated amorphous cellulose, suggesting that more elaborate complexes can be engineered to degrade different types of more complex biomass.

Several studies have shown that B. subtilis can be used as a host to secrete heterologous cellulases, and naturally occurring strains have been identified that secrete cellulases (Himmel et al. (2007) Science 315: 804-807; Joliff et al. (1989) Appl. Environ. Microbiol. 55: 2739-2744; Rincon et al. (2005) J. Bacteriol. 187: 7569-7578; Stephenson and Harwood (1998) Appl. Environ. Microbiol. 64: 2875-2881). Cellulase enzymes have also been targeted to the membrane, enabling protoplasts of B. subtilis to degrade CMC (Kim et al. (2005) Biochem. Biophys. Res. Commun. 334: 1248-1253). However, to the best of our knowledge, this is the first example of a cellulolytic B. subtilis strain in which cellulases and cellulase containing complexes are attached to the peptidoglycan. Interestingly, B. subtilis displaying anchored CelA protein degrades CMC as well as, or better than, two previously described minicellulosomes that contain similar endoglucanases. Direct comparisons are problematic as minicellulosomes can have distinct enzyme components and cellulolytic activity can be measured using a variety of different substrates. However, two previous studies used CMC to measure the activity of a minicellulosome and thus serve as a useful benchmark. Doi and colleagues measured the CMC activity of a purified minicellulosome that contained two copies of the EngB protein bound to a scaffoldin containing two cohesin modules (Cha et al. (2007) J. Microbiol. Biotechnol. 17: 1782-8178). As compared to this system, engineered B. subtilis harboring displaying only the CelA-cohesin complex is ˜4-fold more effective at degrading CMC after an incubation time of 30 minutes. B. subtilis displaying a single enzyme is also slightly more active (˜30%) and more effective at degrading CMC than a previously reported engineered yeast strain (Tsai et al. (2009) Appl. Environ. Microbiol. 75: 6087-6093). Although the cell densities used in this study were not reported, it is tempting to speculate that the elevated levels of cellulase activity in B. subtilis are due to a greater number of complexes being anchored to its cell wall; in yeast only 10,000-100,000 molecules can be displayed via the Aga1-Aga2 interaction used to anchor the minicellulosome (Chao et al. (2006) Nat. Protoc. 1: 755-768).

In conclusion, we have created cellulolytic B. subtilis that contain a minicellulosome covalently attached to the cell wall by a heterologous sortase enzyme. The approaches described herein are expected to yield industrially useful strains that display minicellulosomes with multiple enzymes that synergistically degrade different types of biomass.

The cellulolytic activity of B. subtilis is also quite stable, which is in marked contrast to non-covalently attached cellulosomes in C. thermocellum that detach from the cell as it enters stationary phase (Bayer et al. (1998) J. Struct. Biol. 124: 221-234; Rincon et al. (2005) J. Bacteriol. 187: 7569-7578). Finally, the robust genetic system of B. subtilis makes it feasible to use it as a consolidated bioprocessor in which both cellulolytic and biofuel producing metabolic pathways are genetically introduced into a single microorganism.

### Example 2

### Recombinant B. subtilis Cells that are Capable of Degrading Industrially Relevant Biomass

The protein display system described herein was used to create recombinant B. subtilis cells that are capable of degrading industrially relevant biomass. Example 1 describes the use of the system to display cellulases that are active against acid-treated amorphous cellulose and carboxymethyl cellulose (a methylated, soluble form of cellulose).

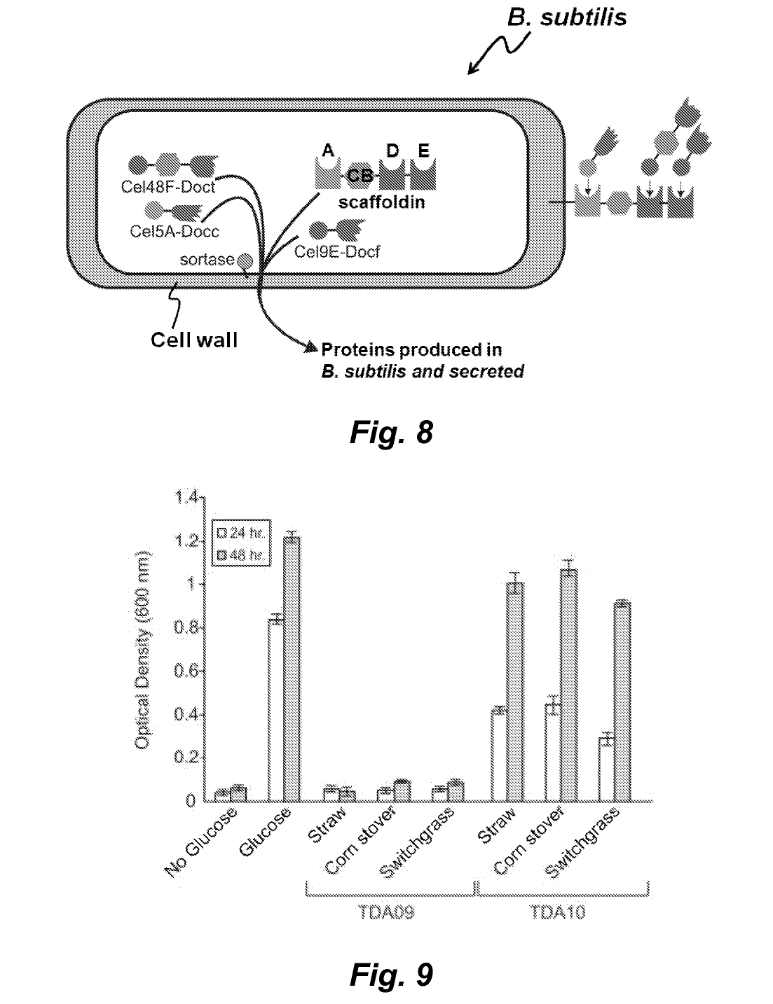

This example describes engineering of the display system to degrade biomass. The reengineered cells display the minicellulosome shown in FIG. 8 and enable B. subtilis to degrade biomass, a capability that is lacking in native strains of this microbe. This was accomplished by displaying minicellulosomes that incorporate a different set of cellulase enzymes. The enzymes that are displayed include the Clostridium cellulolyticum endoglucanase Cel5A, Clostridium cellulolyticum endoglucanase Cel48F and the C. cellulolyticum exoglucanase Cel9E. These enzymes were chosen because they have previously been shown to degrade biomass (Fierobe et al. (2005) J. Biol. Chem. 280: 16325-16334). In order to simplify the expression of the components necessary to assemble the cellulolytic complexes we also eliminated the need to add xylose (a potential carbon nutrient source). In the present system the genes encoding the sortase, scaffoldin and three cellulase enzymes are induced using a single inductant, IPTG. However, it should be emphasized that only trivial changes are needed to engineer cells that would constitutively express these proteins and thus eliminate the need for IPTG.

Data demonstrating that minicellulosome displaying B. subtilis degrades biomass is shown in FIG. 9. Two strains were studied initially. The first strain expresses the intact minicellulosome shown in FIG. 1 (strain TDA10), while the second strain is identical to strain TDA10 but does not express the three cellulases (strain TDA09). Cells were grown in an overnight culture to an optical density at 600 nm (OD600) of ˜1.5. A 100 μl aliquot from these cultures was then used to inoculate 5 mL of S7 minimal medium in which the sole carbon source was biomass (either switchgrass, corn stover, or straw). The cells were then shaken at 37° C. and growth was monitored by measuring the OD600 after 24 and 48 hours. Only cells displaying the minicellulosome can grow using biomass as a nutrient (compare strains TDA09 (no cellulases) and minicellulosome displaying strain TDA10). Control experiments were performed to verify that the protein components of the minicellulosome were displayed on the cell wall and that incorporation into the cell wall via the scaffoldin was needed to confer growth on biomass. Moreover, a native strain of B. subtilis which does not display cellulases was shown to be incapable of growth on biomass (data not shown). Importantly, the data indicates that minicellulosome displaying cells can efficiently degrade biomass because when only this substance is provided as a carbon source, they reach optical densities after 48 hours that are comparable to those obtained by native strains of B. subtilis grown using glucose as a nutrient (FIG. 9).

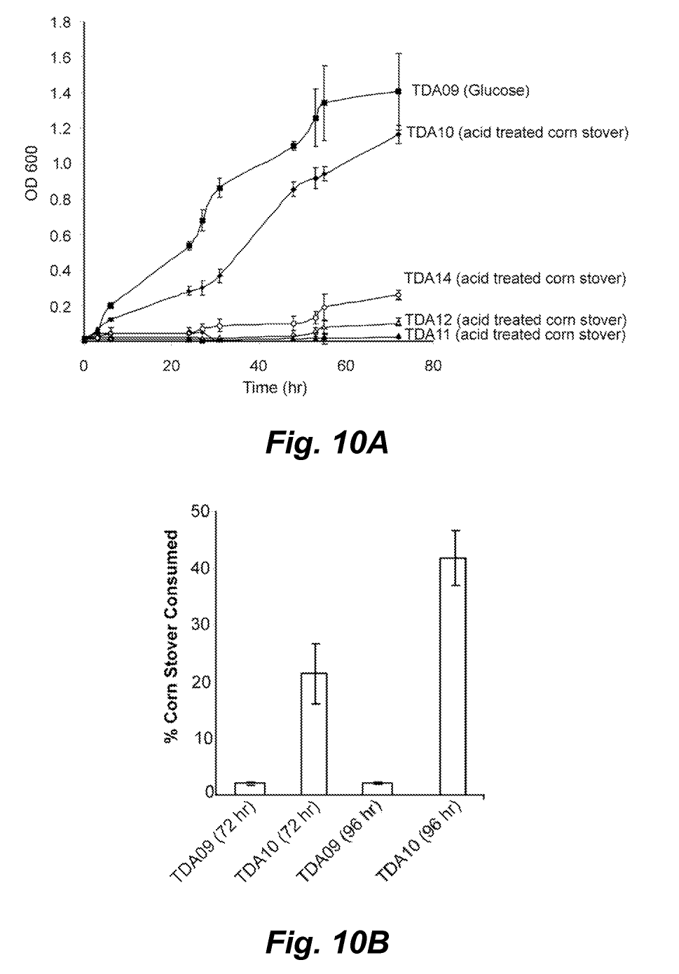

Common procedures used to degrade biomass into component sugars frequently require pretreatment of the biomass with acid which primes it for degradation by cellulases. We therefore determined the ability of the minicellulosome displaying cells to degrade this form of biomass. Corn stover was immersed in 0.8% sulfuric acid, and heated to 120° C. for 30 minutes in a laboratory autoclave. The corn stover was then repeatedly washed with deionized water to neutralize its pH. The ability of this material to be degraded by various strains of B. subtilis was determined using the procedures described above. As shown in FIG. 10A only TDA10 cells displaying the minicellulosome can grow on acid treated corn stover. Importantly, several control experiments indicate that the intact minicellulosome displayed on the cell surface is required for efficient biomass degradation. For example, strain TDA11 is identical to strain TDA10 but does not produce the scaffoldin which coordinates the assembly of the enzymes on the cell surface. As this strain is unable to grow this indicates that the enzymes need to be sequestered on the cell surface to efficiently degrade biomass. Additional studies demonstrate that the full complement of three cellulases (Cel5A, Cel9E and Cel48F) is required for robust growth on biomass. This is shown by the growth behavior of strains TDA12 and TDA14. They are identical to strain TDA10 except that strain TDA12 displays only a single enzyme (Cel9E) and strain TDA14 displays only two enzymes (Cel9E and Cel5A). The finding that these strains grow poorly indicates that at least three enzymes are required for robust growth and is quite promising as it suggests that the display of additional enzyme with different activities could significantly improve biomass degradation even further.

The ability of strain TDA10 to grow when only biomass is provided as a carbon source (FIGS. 9 and 10A) indirectly indicates that these minicellulosome displaying cells degrade biomass and import the component sugars for use as a nutrient (intact biomass is too large to be imported into the cell and degraded). To confirm this hypothesis we directly measured the ability of the cells to degrade biomass. A separate set of experiments were performed as described above, but the degradation of biomass by the cells was determined by measuring the dry weight of the biomass before and after exposure to the cells. FIG. 10B shows the percent biomass consumed after 72 and 96 hours of exposure to the cells. As much as ˜40% of the biomass can be degraded after 96 hours. Importantly, cells not displaying a minicellulosome are unable to decompose the biomass, and little is lost by solubilization of the biomass.

A major goal is to create industrially useful microbes that are capable of degrading biomass and producing biofuels. Towards this objective others have displayed cellulases on various microorganisms using different approaches from those outlined here. However, to the best of our knowledge, none of these recombinant microbes are capable of degrading bona fide biomass into its component sugars. Typical studies only look at cellulose derivatives such as CMC, amorphous cellulose, acid treated avicel. This may be because in these previous studies an insufficient number of enzymes with the appropriate catalytic activities were displayed (Lilly et al. (2009) FEMS Yeast Res., 9:1236-1249; Tsai et al. (2009) Appl. Environ. Microbiol., 75: 6087-6093; Wen et al. (2010) Appl. Environ/Microbiol. 76: 1251-1260). We have overcome this problem because the display systems described herein are quite robust and enable multiple enzymes and multi-enzyme complexes to be presented on the cell surfaced at high density.

The ability of minicellulosome displaying B. subtilis cells to decompose biomass is an important step towards improving biomass degradation processes needed to produce biofuels. It can also facilitate the creation of consolidated bioprocessing organisms that produce biofuels and other important industrial compounds from biomass. Thus, for example, engineered cells engineered using the methods described herein can be used to directly degrade biomass and thereby replace the need to use purified enzyme cocktails (which is currently practiced in cellulosic ethanol plants). This can be accomplished by engineering cells that contain a larger number of distinct enzymes on their surfaces such that the cells have more potent cellulolytic activity. This is a very achievable goal as it seems likely that the number of distinct enzymes displayed on the cell surface can be increased several fold because B. subtilis has a robust genetic system and other microbes produce cellulosome complexes that contain a large number of enzymes. The B. subtilis cells described in this example, consume the biomass as a nutrient. However, if the goal is to degrade biomass into component sugars that can be subsequently used as feed stock for fermentation by another biofuel producing organism (presumably yeast as is currently practiced) it is desirable to maximize biomass conversion. This can be facilitated by genetically engineering the cells (bacteria) described herein to eliminate their ability to import glucose. In practice, these recombinant cells would first be grown to high density on a cheap nutrient source and then applied to the biomass to degrade it into component sugars.

Another application of the display systems described herein is their use as a tool to identify new cellulases or combinations of cellulases that are better able to degrade a particular type of biomass. This is because B. subtilis has a powerful genetic system and we have shown that cell growth is dependent upon cellulase display. Thus, cell growth can be used to select for cells displaying more potent enzymes and/or enzyme complexes. For example, randomly mutated cellulase enzymes could be displayed on the cell surface and the most active enzyme determined by selecting for strains that grow best. Selection approaches could also be used to search for the optimal arrangement, composition and number of enzymes on the cell surface and/or to screen the biomass degrading potential of cellulase expressing genes from other organisms. It is important to note that to the best of our knowledge no other cell-based or in vitro systems exist that can be used to select for enzymes or enzyme complexes with increased activity against biomass. Finally, the system we have developed can be used to create a consolidated bioprocessing organism capable of converting biomass directly into biofuels. In one illustrative embodiment, this can be accomplished by introducing biofuel producing metabolic pathways directly into cellulolytic B. subtilis cells or by porting the minicellulosome structures discovered into the other microbes that produce useful fuels.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

SEQUENCE LISTING
Protein sequences of genes used in Example 1 with
accession codes.
SrtA
B. anthracis Ames SrtA (GenBank accession AE016879.1)
(SEQ ID NO: 40):
MNKQRIYSIVAILLFVVGGVLIGKPFYDGYQAEKKQTENVQAVQKMDYEKHETEFVDASKI
DQPDLAEVANASLDKKQVIGRISIPSVSLELPVLKSSTEKNLLSGAATVKENQVMGKGNYA
LAGHNMSKKGVLFSDIASLKKGDKIYLYDNENEYEYAVTGVSEVTPDKWEVVEDHGKDEIT
LITCVSVKDNSKRYVVAGDLVGTKAKKDYKDDDDK
CelA
B. subtilis 168 PhrC (GenBank accession ZP_03590039)
fused to endoglucanase CelA from C. thermocellum
ATCC27405 (residues 32-434, GenBank accession K03088)
and the C-terminal domain of S. aureus NCTC 8325
(residues 756-914, GenBank accession CP000253)
(SEQ ID NO: 41):
MKLKSKLFVICLAAAAIFTAAGVSANAEALDFHVTTSHHHHHHHAGVPFNTKYPYGPTSIA
DNQSEVTAMLKAEWEDWKSKRITSNGAGGYKRVQRDASTNYDTVSEGMGYGLLLAVCFNEQ
ALFDDLYRYVKSHFNGNGLMHWHIDANNNVTSHDGGDGAATDADEDIALALIFADKLWGSS
GAINYGQEARTLINNLYNHCVEHGSYVLKPGDRWGGSSVTNPSYFAPAWYKVYAQYTGDTR
WNQVADKCYQIVEEVKKYNNGTGLVPDWCTASGTPASGQSYDYKYDATRYGWRTAVDYSWF
GDQRAKANCDMLTKFFARDGAKGIVDGYTIQGSKISNNHNASFIGPVAAASMTGYDLNFAK
ELYRETVAVKDSEYYGYYGNSLRLLTLLYITGNFPNPLSDLSGQPTPPSNPTPSLPPQVVY
GDVNGDGNVNSTDLTMLKRYLLKSVTNINREAADVNRDGAINSSDMTILKRYLIKSIPHLP
YAAAQVSGHNEGQQTIEEDTTPPIVPPTPPTPEVPSEPETPTPPTPEVPSEPETPTPPTPE
VPTEPGKPIPPAKEEPKKPSKPVEQGKVVTPVIEINEKVKAVVPTKKAQSKKSELPETGGE
ESTNNGMLFGGLFSILGLALLRRNKKNHKA-
Coh
B. subtilis 168 PhrC (GenBank accession ZP_03590039)
fused to the second cohesin domain (Coh, residues
182-328, GenBank accession ABN54273) of C.
thermocellum CipA and the C-terminal domain of S.
aureus NCTC 8325 (residues 756-914, GenBank
accession CP000253)
(SEQ ID NO: 42):
MKLKSKLFVICLAAAAIFTAAGVSANAEALDFHVTSRHHHHHHGVVVEIGKVTGSVGTTVE
IPVYFRGVPSKGIANCDFVFRYDPNVLEIIGIDPGDIIVDPNPTKSFDTAIYPDRKIIVFL
FAEDSGTGAYAITKDGVFAKIRATVKSSAPGYITFDEVGGFADNDLVEQKVSFIDGGVNVG
NATPTKGSQVSGHNEGQQTIEEDTTPPIVPPTPPTPEVPSEPETPTPPTPEVPSEPETPTP
PTPEVPTEPGKPIPPAKEEPKKPSKPVEQGKVVTPVIEINEKVKAVVPTKKAQSKKSELPE
TGGEESTNNGMLFGGLFSILGLALLRRNKKNHKA-
CelA-Doct
Endoglucanase CelA from C. thermocellum ATCC 27405
(residues 32-434, GenBank accession K03088) fused
to the carbohydrate binding module and type I
dockerin from C. thermocellum Xyn10B (residues
540-790, GenBank accession ABN52146)
(SEQ ID NO: 43):
MGSSHHHHHHSSGLVPAGSHMASEQKLISEEDLAGVPFNTKYPYGPTSIADNQSEVTAMLK
AEWEDWKSKRITSNGAGGYKRVQRDASTNYDTVSEGMGYGLLLAVCFNEQALFDDLYRYVK
SHFNGNGLMHWHIDANNNVTSHDGGDGAATDADEDIALALIFADKLWGSSGAINYGQEART
LINNLYNHCVEHGSYVLKPGDRWGGSSVTNPSYFAPAWYKVYAQYTGDTRWNQVADKCYQI
VEEVKKYNNGTGLVPDWCTASGTPASGQSYDYKYDATRYGWRTAVDYSWFGDQRAKANCDM
LTKFFARDGAKGIVDGYTIQGSKISNNHNASFIGPVAAASMTGYDLNFAKELYRETVAVKD
SEYYGYYGNSLRLLTLLYITGNFPNPLSDLSGQPTPPSNPTPSLPPQVVYGDVNGDGNVNS
TDLTMLKRYLLKSVTNINREAADVNRDGAINSSDMTILKRYLIKSIPHLPYAAAQSEWGDG
NNPAGGGGGGKPEEPDANGYYYHDTFEGSVGQWTARGPAEVLLSGRTAYKGSESLLVRNRT
AAWNGAQRALNPRTFVPGNTYCFSVVASFIEGASSTTFCMKLQYVDGSGTQRYDTIDMKTV
GPNQWVHLYNPQYRIPSDATDMYVYVETADDTINFYIDEAIGAVAGTVIEGPAPQPTQPPV
LLGDVNGDGTINSTDLTMLKRSVLRAITLTDDAKARADVDKNGSINSTDVLLLSRYLLRVI
DKFPVAENP-
Scaf
B. subtilis 168 PhrC (GenBank accession ZP_03590039)
fused to a type I cohesin from C. cellulolyticum CipC
(residues 347-482, GenBank accession U40345), a type
I cohesin and carbohydrate binding module from C.
thermocellum CipA (residues 391-927, GenBank accession
L08665) and a type I cohesin from R. flavefaciens ScaB
(residues 90-246, GenBank accession AJ278969) and he
C-terminal domain of S. aureus NCTC 8325 (residues
756-914, GenBank accession CP000253)
(SEQ ID NO: 44):
MKLKSKLFVICLAAAAIFTAAGVSANAEALDFHVTTSYPYDVPDYDSLKVTVGTANGKPGD
TVTVPVTFADVAKMKNVGTCNFYLGYDASLLEVVSVDAGPIVKNAAVNFSSSASNGTISFL
FLDNTITDELITADGVFANIKFKLKSVTAKTTTPVTFKDGGAFGDGTMSKIASVTKTNGSV
RSPTKSATATPTRPSVPTNTPTNTPANTPVSGNLKVEFYNSNPSDTTNSINPQFKVTNTGS
SAIDLSKLTLRYYYTVDGQKDQTFWCDHAAIIGSNGSYNGITSNVKGTFVKMSSSTNNADT
YLEISFTGGTLEPGAHVQIQGRFAKNDWSNYTQSNDYSFKSASQFVEWDQVTAYLNGVLVW
GKEPGGSVVPSTQPVTTPPATTKPPATTKPPATTIPPSDDPNAIKIKVDTVNAKPGDTVNI
PVRFSGIPSKGIANCDFVYSYDPNVLEIIEIKPGELIVDPNPDKSFDTAVYPDRKIIVFLF
AEDSGTGAYAITKDGVFATIVAKVKSGAPNGLSVIKFVEVGGFANNDLVEQRTQFFDGGVN
VGDGSAGGLSAVQPNVSLGEVLDVSANRTAADGTVEWLIPTVTAAPGQTVTMPVVVKSSSL
AVAGAQFKIQAATGVSYSSKTDGDAYGSGIVYNNSKYAFGQGAGRGIVAADDSVVLTLAYT
VPADCAEGTYDVKWSDAFVSDTDGQNITSKVTLTDGAIIVKHAAAQVSGHNEGQQTIEEDT
TPPIVPPTPPTPEVPSEPETPTPPTPEVPSEPETPTPPTPEVPTEPGKPIPPAKEEPKKPS
KPVEQGKVVTPVIEINEKVKAVVPTKKAQSKKSELPETGGEESTNNGMLFGGLFSILGLAL
LRRNKKNHKA-
CelE-Docc
Exoglucanse CelE (residues 29-885, GenBank accession
M87018) of C. cellulolyticum
(SEQ ID NO: 45):
MHHHHHHKKRLVKKVAMLIAIVLVLSSSIGQAFALVGAGDLIRNHTFDNRVGLPWHVVESY
PAKASFEITSDGKYKITAQKIGEAGKGERWDIQFRHRGLALQQGHTYTVKFTVTASRACKI
YPKIGDQGDPYDEYWNMNQQWNFLELQANTPKTVTQTFTQTKGDKKNVEFAFHLAPDKTTS
EAQNPASFQPITYTFDEIYIQDPQFAGYTEDPPEPTNVVRLNQVGFYPNADKIATVATSST
TPINWQLVNSTGAAVLTGKSTVKGADRASGDNVHIIDFSSYTTPGTDYKIVTDVSVTKAGD
NESMKFNIGDDLFTQMKYDSMKYFYHNRSAIPIQMPYCDQSQWARPAGHTTDILAPDPTKD
YKANYTLDVTGGWYDAGDHGKYVVNGGIATWTVMNAYERALHMGGDTSVAPFKDGSLNIPE
SGNGYPDILDEARYNMKTLLNMQVPAGNELAGMAHHKAHDERWTALAVRPDQDTMKRWLQP
PSTAATLNLAAIAAQSSRLWKQFDSAFATKCLTAAETAWDAAVAHPEIYATMEQGAGGGAY
GDNYVLDDFYWAACELYATTGSDKYLNYIKSSKHYLEMPTELTGGENTGITGAFDWGCTAG
MGTITLALVPTKLPAADVATAKANIQAAADKFISISKAQGYGVPLEEKVISSPFDASVVKG
FQWGSNSFVINEAIVMSYAYEFSDVNGTKNNKYINGALTAMDYLLGRNPNIQSYITGYGDN
PLENPHHRFWAYQADNTFPKPPPGCLSGGPNSGLQDPWVKGSGWQPGERPAEKCFMDNIES
WSTNEITINWNAPLVWISAYLDEKGPEIGGSVTPPTNLGDVNGDGNKDALDFAALKKALLS
QDTSTINVANADINKDGSIDAVDFALLKSFLLGKITL
CelG-Docf
Endoglucanase CelG (residues 51-654, GenBank accession
M87018) fused to a type I dockerin from R. flavefaciens
ScaA (residues 789-879, GenBank accession AJ278969)
(SEQ ID NO: 46):
FNLPMSYTSAMLAWSLYEDKDAYDKSGQTKYIMDGIKWANDYFIKCNPTPGVYYYQVGDGG
KDHSWWGPAEVMQMERPSFKVDASKPGSAVCASTAASLASAAVVFKSSDPTYAEKCISHAK
NLFDMADKAKSDAGYTAASGYYSSSSFYDDLSWAAVWLYLATNDSTYLDKAESYVPNWGKE
QQTDIIAYKWGQCWDDVHYGAELLLAKLTNKQLYKDSIEMNLDFWTTGVNGTRVSYTPKGL
AWLFQWGSLRHATTQAFLAGVYAEWEGCTPSKVSVYKDFLKSQIDYALGSTGRSFVVGYGV
NPPQHPHHRTAHGSWTDQMTSPTYHRHTIYGALVGGPDNADGYTDEINNYVNNEIACDYNA
GFTGALAKMYKHSGGDPIPNFKAIEKITNDEVIIKAGLNSTGPNYTEIKAVVYNQTGWPAR
VTDKISFKYFMDLSEIVAAGIDPLSLVTSSNYSEGKNTKVSGVLPWDVSNNVYYVNVDLTG
ENIYPGGQSACRREVQFRIAAPQGRRYWNPKNDFSYDGLPTTSTVNTVTNIPVYDNGVKVF
GNEPEPAGGSEPGTKLVPTWGDTNCDGVVNVADVVVLNRFLNDPTYSNITDQGKVNADVVD
PQDKSGAAVDPAGVKLTVADSEAILKAIVELITLPQLE

1. A recombinant modified Gram-positive bacterium that displays on its surface one or more cellulolytic enzymes, said bacterium comprises:
i) a protein comprising one or more cellulolytic enzymes covalently linked to the surface of said bacterium, and a nucleic acid construct that encodes said protein and comprising one or more cellulolytic enzymes attached to a secretory signal sequence at the N-terminus of said protein and a cell wall sorting signal at the carboxyl terminus of said protein; and/or
ii) a protein comprising one or more cohesin domains covalently linked to the surface of said bacterium, wherein said bacterium, comprises a nucleic acid construct that encodes said protein comprising said one or more cohesin domains attached to a secretory signal sequence at the N-terminus of said protein and a cell wall sorting signal at the carboxyl terminus of said protein.

2. The bacterium of claims 1, wherein said cell wall sorting signal comprises:
a motif selected from the group consisting of LPXTG (SEQ ID NO:14), NPQTN (SEQ ID NO:13), LPXTGG (SEQ ID NO: 18), LPXTA(ST) (SEQ ID NO:19), and LAXTG (SEQ ID NO:20) where X is any amino acid; and/or
a cell wall sorting signal from a Gram-positive microorganism that encodes an endogenous sortase enzyme; and/or
comprises a cell wall sorting signal from an organism selected from the group consisting of S. aureus, S. sobrinus, E. faecalis, S. pyogenes, L. monocytogenes, A. viscosus, S. agalactiae, S. aureus, S. mutans, and S. pyogenes; and/or
a domain of the Staphylococcus aureus Fibronectin Binding Protein B; and/or
an amino acid sequence selected from the group consisting of
(SEQ ID NO: 1)
LPETGGEESTNNGMLFGGLFSILGLALLRRNKKNHKA,
(SEQ ID NO: 2)
LPETGEENPFI GTTVFGGLSLALGAALLAGRRREL,
(SEQ ID NO: 3)
LPETGGEES TNKGMLF GGLF S I LGLALLRRNKKNHKA,
(SEQ ID NO: 4)
LP AT GDS SNAYLPLLGLVS LT AGFS LLGLRRKQD,
(SEQ ID NO: 5)
LP KTGEKQNVLLTVVGS LAAMLGLAGLGFKRRKETK,
(SEQ ID NO: 6)
LP S TGS I GT YLF KAI GS AAMI GAI GI YI
VKRRKA,
(SEQ ID NO: 7)
LPTTGDSDNALYLLLGLLAVGTAMALT KKARAS K,
(SEQ ID NO: 8)
LPLTGANGVI FLTI AGALLVAGGAVVAYANKRRHVAKH,
(SEQ ID NO: 9)
LPYTGVAS NLVLEI MGLLGLI GTS F I AMKRRKS,
(SEQ ID NO: 10)
LPKTGMKI I TS WI TWVF I GI LGLYLI LRKRFNS,
(SEQ ID NO: 11)
LPSTGEQAGLLLTTVGLVI VAVAGVYF YRTRR,
and
(SEQ ID NO: 12)
LP S TGETANPFFTAAALTVMATAGVAAVVKRKEEN.

3-7. (canceled)

8. The bacterium of claim 1, wherein said bacterium encodes an endogenous sortase transpeptidase.

9. (canceled)

10. The bacterium claim 1, wherein said bacterium further comprises a construct encoding a sortase transpeptidase.

11. The bacterium of claim 10, wherein said sortase transpeptidase is a sortase A enzyme or a homologue thereof.

12-15. (canceled)

16. The bacterium of claim 1, wherein said secretory signal sequence comprises the amino acid sequence
(SEQ ID NO: 13)
MKLKSKLFVICLAAAAIFTAAGVS ANAE ALDFHVT.

17. The bacterium of claim 1, wherein one or more endogenous proteases of said bacterium are down-regulated or knocked out.

18. The bacterium of claim 17, wherein said one or more endogenous proteases comprise a cell wall protease.

19. (canceled)

20. The bacterium of claim 1, wherein said bacterium comprises a protein comprising one or more cellulolytic enzymes covalently linked to the surface of said microorganism, and a nucleic acid construct that encodes said protein and one or more cellulolytic enzymes attached to a secretory signal sequence at the N-terminus of said protein and a cell wall sorting signal at the carboxyl terminus of said protein.

21. The bacterium of claim 20, wherein said protein further comprises a cellulose or carbohydrate binding domain (CBD).

22. The bacterium of claim 1, wherein said bacterium comprises a protein comprising one or more cohesin domains covalently linked to the surface of said microorganism, wherein said bacterium, comprises a nucleic acid construct that encodes said protein comprising said one or more cohesin domains attached to a secretory signal sequence at the N-terminus of said protein and a cell wall sorting signal at the carboxyl terminus of said protein.

23. The bacterium of claim 22, wherein one or more of said cohesin domains are attached to one or more proteins comprising a dockerin attached to one or more cellulolytic enzymes wherein said dockerin is bound to said one or more cohesin domains.

24. The bacterium of claim 23, wherein said protein comprising a dockerin attached to a cellulolytic enzyme further comprises a cellulose or carbohydrate binding domain (CBD).

25-26. (canceled)

27. The bacterium of claim 22, wherein:
said dockerin attached to a cellulolytic enzyme is encoded by a construct in said bacterium; or
said dockerin attached to a cellulolytic enzyme is provided from a source extrinsic to said bacterium.

28. (canceled)

29. The bacterium of claim 1, wherein said cellulolytic enzyme(s) on dormant bacteria are stable for at least 1 day, more preferably for at least 2 days, and most preferably at least 3 days.

30. The bacterium of claim 1, wherein said cellulolytic enzyme(s) comprise one or more enzymes selected from the group consisting of an endocellulase, an exocellulase, a beta-glucosidase (cellobiase), an oxidative cellulase, a xylanase, a hemicellulase, a lichenase, a chitenase, and a cellulose phosphorylase.

31. The bacterium of claim 23, wherein a plurality of cellulolytic enzymes are present forming a minicellulosome.

32. The bacterium of claim 31, wherein said minicellulosome comprises at least 2, different enzymes.

33. (canceled)

34. The bacterium of claim 31, wherein said minicellulosome comprises:
at least one endoglucanase; and/or
at least one exoglucanase; and/or
at least two endoglucanases and at least one exoglucanase; and/or
Clostridium cellulolyticum endoglucanase Cel5A, C. cellulolyticum endoglucanase Cel48F, and C. cellulolyticum exoglucanase Cel9E.

35-39. (canceled)

40. The bacterium of claim 39, wherein said Gram-positive bacterium comprises a genus selected from the group consisting of Corynebacterium, Clostridium, Listeria, thermophilic Geobacillus, and Bacillus.

41-45. (canceled)

46. A method of degrading cellulosic biomass into fermentable sugars, said method comprising:
contacting said cellulosic biomass with a bacterium of claim 1, under conditions in which said bacteria partially or fully degrade cellulose in said cellulosic biomass to form one or more fermentable sugars.

47-50. (canceled)

51. A consolidated bioreactor for the conversion of a lignocellulosic biomass into bioethanol said bioreactor comprising:
a culture system that cultures bacteria of claim 1 under conditions in which said bacteria partially or fully degrade cellulose in said lignocellulosic biomass to form one or more fermentable sugars; and
a culture system that ferments said sugars to form a biofuel.

52. An isolated nucleic acid that encodes:
a protein comprising one or more cellulolytic enzymes attached to a secretory signal sequence at the N-terminus of said protein and a cell wall sorting signal at the carboxyl terminus of said protein; and/or
a protein comprising one or more cohesin domains attached to a secretory signal sequence at the N-terminus of said protein and a cell wall sorting signal at the carboxyl terminus of said protein.

53-61. (canceled)

62. A vector comprising a nucleic acid of claim 52 operably linked to a promoter.

63-65. (canceled)

66. A method of identifying cellulolytic enzyme combinations that enhance degradation of a particular substrate said method comprising:
providing a plurality of recombinant bacteria of claim 1, wherein said bacteria each display at least two cellulolytic enzymes and different bacteria display different enzymes;
contacting said substrate with said bacteria; and
selecting bacteria that show enhanced degradation of said substrate and/or improved growth on said substrate.

67. A method of identifying cellulolytic enzyme variants that enhance degradation of a particular substrate said method comprising:
providing a plurality of recombinant bacteria of claim 1, wherein said bacteria each display at least one cellulolytic enzyme variant and different bacteria display different cellulolytic enzyme variants;
contacting said substrate with said bacteria; and
selecting bacteria that show enhanced degradation of said substrate and/or improved growth on said substrate.

68-70. (canceled)


SEQUENCE LISTING (46 sequences)

SEQ ID NO: 1 - protein, 37 aa, Artificial
  Sorting signal peptide
LPETGGEESTNNGMLFGGLFSILGLALLRRNKKNHKA

SEQ ID NO: 2 - protein, 35 aa, Artificial
  Sorting signal peptide
LPETGEENPFIGTTVFGGLSLALGAALLAGRRREL

SEQ ID NO: 3 - protein, 37 aa, Artificial
  Sorting signal peptide
LPETGGEESTNKGMLFGGLFSILGLALLRRNKKNHKA

SEQ ID NO: 4 - protein, 34 aa, Artificial
  Sorting signal peptide
LPATGDSSNAYLPLLGLVSLTAGFSLLGLRRKQD

SEQ ID NO: 5 - protein, 36 aa, Artificial
  Sorting signal peptide
LPKTGEKQNVLLTVVGSLAAMLGLAGLGFKRRKETK

SEQ ID NO: 6 - protein, 34 aa, Artificial
  Sorting signal peptide
LPSTGSIGTYLFKAIGSAAMIGAIGIYIVKRRKA

SEQ ID NO: 7 - protein, 34 aa, Artificial
  Sorting signal peptide
LPTTGDSDNALYLLLGLLAVGTAMALTKKARASK

SEQ ID NO: 8 - protein, 38 aa, Artificial
  Sorting signal peptide
LPLTGANGVIFLTIAGALLVAGGAVVAYANKRRHVAKH

SEQ ID NO: 9 - protein, 33 aa, Artificial
  Sorting signal peptide
LPYTGVASNLVLEIMGLLGLIGTSFIAMKRRKS

SEQ ID NO: 10 - protein, 33 aa, Artificial
  Sorting signal peptide
LPKTGMKIITSWITWVFIGILGLYLILRKRFNS

SEQ ID NO: 11 - protein, 32 aa, Artificial
  Sorting signal peptide
LPSTGEQAGLLLTTVGLVIVAVAGVYFYRTRR

SEQ ID NO: 12 - protein, 35 aa, Artificial
  Sorting signal peptide
LPSTGETANPFFTAAALTVMATAGVAAVVKRKEEN

SEQ ID NO: 13 - protein, 35 aa, Artificial
  Sorting signal peptide
MKLKSKLFVICLAAAAIFTAAGVSANAEALDFHVT

SEQ ID NO: 14 - protein, 5 aa, Artificial
  Sorting signal motif
LPXTG

SEQ ID NO: 15 - protein, 5 aa, Staphylococcus aureus
GGGGG

SEQ ID NO: 16 - protein, 6 aa, Artificial
  Affinity tag
HHHHHH

SEQ ID NO: 17 - protein, 5 aa, Artificial
  Sorting signal motif
NPQTN

SEQ ID NO: 18 - protein, 6 aa, Artificial
  Sorting signal motif
LPXTGG

SEQ ID NO: 19 - protein, 7 aa, Artificial
  Sorting signal motif
LPXTAST

SEQ ID NO: 20 - protein, 5 aa, Artificial
  Sorting signal motif
LAXTG

SEQ ID NO: 21 - DNA, 52 bp, Artificial
  Oligonucleotide primer
gacagaagcttaaaggagtgaaggtttgtatgaataagcaaagaatttatag

SEQ ID NO: 22 - DNA, 47 bp, Artificial
  Oligonucleotide primer
gacagggatccttatttatcatcatcatctttataatctttcttcgc

SEQ ID NO: 23 - DNA, 32 bp, Artificial
  Oligonucleotide primer
gacagaagcttaaaggagtgaaggtttgtatg

SEQ ID NO: 24 - DNA, 31 bp, Artificial
  Oligonucleotide primer
gacagactagttgtcacatgaaagtcgagtg

SEQ ID NO: 25 - DNA, 59 bp, Artificial
  Oligonucleotide primer
gacagactagtcatcatcatcatcatcatcatgcaggtgtgccttttaacacaaaatac

SEQ ID NO: 26 - DNA, 33 bp, Artificial
  Oligonucleotide primer
gacaggcggccgcataaggtaggtggggtatgc

SEQ ID NO: 27 - DNA, 39 bp, Artificial
  Oligonucleotide primer
gacaggcggccgcccaagtaagtggtcataatgaaggtc

SEQ ID NO: 28 - DNA, 41 bp, Artificial
  Oligonucleotide primer
gacaggcatgcttatgctttgtgattctttttatttctgcg

SEQ ID NO: 29 - DNA, 49 bp, Artificial
  Oligonucleotide primer
gacagactagtcatcatcatcatcatcatggtgtggtagtagaaattgg

SEQ ID NO: 30 - DNA, 33 bp, Artificial
  Oligonucleotide primer
gacaggcggccgccttggtcggtgttgcattgc

SEQ ID NO: 31 - DNA, 37 bp, Artificial
  Oligonucleotide primer
gacaggcggccgcccagtccgaatggggcgacggtaa

SEQ ID NO: 32 - DNA, 32 bp, Artificial
  Oligonucleotide primer
gacaggcatgcttaaggattttctgctacagg

SEQ ID NO: 33 - DNA, 37 bp, Artificial; identical to SEQ ID NO: 31
  Oligonucleotide primer

SEQ ID NO: 34 - DNA, 30 bp, Artificial
  Oligonucleotide primer
gacagctagcgcaggtgtgccttttaacac

SEQ ID NO: 35 - DNA, 47 bp, Artificial
  Oligonucleotide primer
gaaataaaaagaatcacaaagcaatgtcccctatactaggttattgg

SEQ ID NO: 36 - DNA, 33 bp, Artificial
  Oligonucleotide primer
gacaggcatgcttagtcacgatgcggccgctcg

SEQ ID NO: 37 - DNA, 47 bp, Artificial
  Oligonucleotide primer
ccaataacctagtataggggacattgctttgtgattctttttatttc

SEQ ID NO: 38 - DNA, 60 bp, Artificial
  Oligonucleotide primer
gacagactagttacccatacgatgttccagattacgctggcgattctcttaaagttacag

SEQ ID NO: 39 - DNA, 32 bp, Artificial
  Oligonucleotide primer
gaacaggcggccgccttaacaatgatagcgcc

SEQ ID NO: 40 - protein, 218 aa, Bacillus anthracis
MNKQRIYSIVAILLFVVGGVLIGKPFYDGYQAEKKQTENVQAVQKMDYEKHETEFVDASKIDQPDLAEVANASLDKKQVIGRISIPSVSLELPVLKSSTEKNLLSGAATVKENQVMGKGNYALAGHNMSKKGVLFSDIASLKKGDKIYLYDNENEYEYAVTGVSEVTPDKWEVVEDHGKDEITLITCVSVKDNSKRYVVAGDLVGTKAKKDYKDDDDK

SEQ ID NO: 41 - protein, 640 aa, Bacillus subtilis
MKLKSKLFVICLAAAAIFTAAGVSANAEALDFHVTTSHHHHHHHAGVPFNTKYPYGPTSIADNQSEVTAMLKAEWEDWKSKRITSNGAGGYKRVQRDASTNYDTVSEGMGYGLLLAVCFNEQALFDDLYRYVKSHFNGNGLMHWHIDANNNVTSHDGGDGAATDADEDIALALIFADKLWGSSGAINYGQEARTLINNLYNHCVEHGSYVLKPGDRWGGSSVTNPSYFAPAWYKVYAQYTGDTRWNQVADKCYQIVEEVKKYNNGTGLVPDWCTASGTPASGQSYDYKYDATRYGWRTAVDYSWFGDQRAKANCDMLTKFFARDGAKGIVDGYTIQGSKISNNHNASFIGPVAAASMTGYDLNFAKELYRETVAVKDSEYYGYYGNSLRLLTLLYITGNFPNPLSDLSGQPTPPSNPTPSLPPQVVYGDVNGDGNVNSTDLTMLKRYLLKSVTNINREAADVNRDGAINSSDMTILKRYLIKSIPHLPYAAAQVSGHNEGQQTIEEDTTPPIVPPTPPTPEVPSEPETPTPPTPEVPSEPETPTPPTPEVPTEPGKPIPPAKEEPKKPSKPVEQGKVVTPVIEINEKVKAVVPTKKAQSKKSELPETGGEESTNNGMLFGGLFSILGLALLRRNKKNHKA

SEQ ID NO: 42 - protein, 339 aa, Bacillus subtilis
MKLKSKLFVICLAAAAIFTAAGVSANAEALDFHVTSRHHHHHHGVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNVGNATPTKGSQVSGHNEGQQTIEEDTTPPIVPPTPPTPEVPSEPETPTPPTPEVPSEPETPTPPTPEVPTEPGKPIPPAKEEPKKPSKPVEQGKVVTPVIEINEKVKAVVPTKKAQSKKSELPETGGEESTNNGMLFGGLFSILGLALLRRNKKNHKA

SEQ ID NO: 43 - protein, 741 aa, Clostridium thermocellum
MGSSHHHHHHSSGLVPAGSHMASEQKLISEEDLAGVPFNTKYPYGPTSIADNQSEVTAMLKAEWEDWKSKRITSNGAGGYKRVQRDASTNYDTVSEGMGYGLLLAVCFNEQALFDDLYRYVKSHFNGNGLMHWHIDANNNVTSHDGGDGAATDADEDIALALIFADKLWGSSGAINYGQEARTLINNLYNHCVEHGSYVLKPGDRWGGSSVTNPSYFAPAWYKVYAQYTGDTRWNQVADKCYQIVEEVKKYNNGTGLVPDWCTASGTPASGQSYDYKYDATRYGWRTAVDYSWFGDQRAKANCDMLTKFFARDGAKGIVDGYTIQGSKISNNHNASFIGPVAAASMTGYDLNFAKELYRETVAVKDSEYYGYYGNSLRLLTLLYITGNFPNPLSDLSGQPTPPSNPTPSLPPQVVYGDVNGDGNVNSTDLTMLKRYLLKSVTNINREAADVNRDGAINSSDMTILKRYLIKSIPHLPYAAAQSEWGDGNNPAGGGGGGKPEEPDANGYYYHDTFEGSVGQWTARGPAEVLLSGRTAYKGSESLLVRNRTAAWNGAQRALNPRTFVPGNTYCFSVVASFIEGASSTTFCMKLQYVDGSGTQRYDTIDMKTVGPNQWVHLYNPQYRIPSDATDMYVYVETADDTINFYIDEAIGAVAGTVIEGPAPQPTQPPVLLGDVNGDGTINSTDLTMLKRSVLRAITLTDDAKARADVDKNGSINSTDVLLLSRYLLRVIDKFPVAENP

SEQ ID NO: 44 - protein, 864 aa, Bacillus subtilis
MKLKSKLFVICLAAAAIFTAAGVSANAEALDFHVTTSYPYDVPDYDSLKVTVGTANGKPGDTVTVPVTFADVAKMKNVGTCNFYLGYDASLLEVVSVDAGPIVKNAAVNFSSSASNGTISFLFLDNTITDELITADGVFANIKFKLKSVTAKTTTPVTFKDGGAFGDGTMSKIASVTKTNGSVRSPTKSATATPTRPSVPTNTPTNTPANTPVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEPGGSVVPSTQPVTTPPATTKPPATTKPPATTIPPSDDPNAIKIKVDTVNAKPGDTVNIPVRFSGIPSKGIANCDFVYSYDPNVLEIIEIKPGELIVDPNPDKSFDTAVYPDRKIIVFLFAEDSGTGAYAITKDGVFATIVAKVKSGAPNGLSVIKFVEVGGFANNDLVEQRTQFFDGGVNVGDGSAGGLSAVQPNVSLGEVLDVSANRTAADGTVEWLIPTVTAAPGQTVTMPVVVKSSSLAVAGAQFKIQAATGVSYSSKTDGDAYGSGIVYNNSKYAFGQGAGRGIVAADDSVVLTLAYTVPADCAEGTYDVKWSDAFVSDTDGQNITSKVTLTDGAIIVKHAAAQVSGHNEGQQTIEEDTTPPIVPPTPPTPEVPSEPETPTPPTPEVPSEPETPTPPTPEVPTEPGKPIPPAKEEPKKPSKPVEQGKVVTPVIEINEKVKAVVPTKKAQSKKSELPETGGEESTNNGMLFGGLFSILGLALLRRNKKNHKA

SEQ ID NO: 45 - protein, 891 aa, Clostridium cellulolyticum
MHHHHHHKKRLVKKVAMLIAIVLVLSSSIGQAFALVGAGDLIRNHTFDNRVGLPWHVVESYPAKASFEITSDGKYKITAQKIGEAGKGERWDIQFRHRGLALQQGHTYTVKFTVTASRACKIYPKIGDQGDPYDEYWNMNQQWNFLELQANTPKTVTQTFTQTKGDKKNVEFAFHLAPDKTTSEAQNPASFQPITYTFDEIYIQDPQFAGYTEDPPEPTNVVRLNQVGFYPNADKIATVATSSTTPINWQLVNSTGAAVLTGKSTVKGADRASGDNVHIIDFSSYTTPGTDYKIVTDVSVTKAGDNESMKFNIGDDLFTQMKYDSMKYFYHNRSAIPIQMPYCDQSQWARPAGHTTDILAPDPTKDYKANYTLDVTGGWYDAGDHGKYVVNGGIATWTVMNAYERALHMGGDTSVAPFKDGSLNIPESGNGYPDILDEARYNMKTLLNMQVPAGNELAGMAHHKAHDERWTALAVRPDQDTMKRWLQPPSTAATLNLAAIAAQSSRLWKQFDSAFATKCLTAAETAWDAAVAHPEIYATMEQGAGGGAYGDNYVLDDFYWAACELYATTGSDKYLNYIKSSKHYLEMPTELTGGENTGITGAFDWGCTAGMGTITLALVPTKLPAADVATAKANIQAAADKFISISKAQGYGVPLEEKVISSPFDASVVKGFQWGSNSFVINEAIVMSYAYEFSDVNGTKNNKYINGALTAMDYLLGRNPNIQSYITGYGDNPLENPHHRFWAYQADNTFPKPPPGCLSGGPNSGLQDPWVKGSGWQPGERPAEKCFMDNIESWSTNEITINWNAPLVWISAYLDEKGPEIGGSVTPPTNLGDVNGDGNKDALDFAALKKALLSQDTSTINVANADINKDGSIDAVDFALLKSFLLGKITL

SEQ ID NO: 46 - protein, 648 aa, Ruminococcus flavefaciens
FNLPMSYTSAMLAWSLYEDKDAYDKSGQTKYIMDGIKWANDYFIKCNPTPGVYYYQVGDGGKDHSWWGPAEVMQMERPSFKVDASKPGSAVCASTAASLASAAVVFKSSDPTYAEKCISHAKNLFDMADKAKSDAGYTAASGYYSSSSFYDDLSWAAVWLYLATNDSTYLDKAESYVPNWGKEQQTDIIAYKWGQCWDDVHYGAELLLAKLTNKQLYKDSIEMNLDFWTTGVNGTRVSYTPKGLAWLFQWGSLRHATTQAFLAGVYAEWEGCTPSKVSVYKDFLKSQIDYALGSTGRSFVVGYGVNPPQHPHHRTAHGSWTDQMTSPTYHRHTIYGALVGGPDNADGYTDEINNYVNNEIACDYNAGFTGALAKMYKHSGGDPIPNFKAIEKITNDEVIIKAGLNSTGPNYTEIKAVVYNQTGWPARVTDKISFKYFMDLSEIVAAGIDPLSLVTSSNYSEGKNTKVSGVLPWDVSNNVYYVNVDLTGENIYPGGQSACRREVQFRIAAPQGRRYWNPKNDFSYDGLPTTSTVNTVTNIPVYDNGVKVFGNEPEPAGGSEPGTKLVPTWGDTNCDGVVNVADVVVLNRFLNDPTYSNITDQGKVNADVVDPQDKSGAAVDPAGVKLTVADSEAILKAIVELITLPQLE